(19) United States
(12) Patent Application Publication
(10) Pub. No.: US 2024/0173503 A1
(43) Pub. Date: May 30, 2024

(54) AIRWAY SECUREMENT APPARATUS AND METHOD FOR USE IN STABLE ENVIRONMENTS

(71) Applicant: Securisyn Medical, LLC, Littleton, CO (US)

(72) Inventors: Arthur Kanowitz, Littleton, CO (US); Katie McIntyre, Denver, CO (US); Nam Trinh, Highlands Ranch, CO (US); Mark Bruning, Monument, CO (US)

(21) Appl. No.: 18/284,574

(22) PCT Filed: Apr. 24, 2022
(86) PCT No.: PCT/US22/26081

§ 371 Date: Sep. 28, 2023

Related U.S. Application Data
(60) Provisional application No. 63/180,017, filed on Apr. 26, 2021.

(51) Int. Cl. A61M 16/04; A61M 16/06

(52) U.S. Cl. CPC A61M 16/0497; A61M 16/0688; A61M 2209/088

(57) ABSTRACT

An adjustable airway securement apparatus and method that enables precise, safe and effective positioning and maintenance of an airway device or endotracheal tube apparatus (ETT) in airways of patients in low-risk stable environments. The system is easy to install on and remove from a patient, while at the same time being sufficiently robust to maintain the airway device in its preferred position in a patient's trachea and to prevent clinically significant movement thereof. If a patient must be moved and a more robust airway securement and stabilization system is needed, the more robust system is sequentially installed on the patient in conjunction with corresponding sequential removal of the less robust apparatus while maintaining a continuous and secure airway and ventilation of the patient.

### CROSS REFERENCE TO RELATED APPLICATION

This application claims priority to U.S. Provisional Patent Application Ser. No. 63/180,017 filed on Apr. 26, 2021, the entire contents of which are incorporated herein by reference.

### FIELD OF THE INVENTION

The present invention relates generally to medical apparatus. Specifically, the present invention relates to an airway securement apparatus designed to maintain an airway device in a preselected position in the trachea of a patient and to prevent clinically significant movement thereof and unintentional extubation of the patient. More specifically, the present invention relates to an adjustable airway securement apparatus that enables precise, safe and effective positioning and maintenance of an airway device or endotracheal tube apparatus (ETT) in airways of patients in stable environments, such as in the operating room and while transitioning therefrom to a less stable environment.

### BACKGROUND OF THE INVENTION

Endotracheal intubation is a medical procedure used to place an airway device (artificial airway) into a patient's trachea or airway. The use of an airway device is mandated in situations where an individual, or an animal in veterinary applications, is unable to independently sustain the natural breathing function or maintain an open airway due to unconsciousness, trauma, disease, drugs or anesthesia. Thus, life-saving mechanical ventilation is provided through the airway device which may be in the form of an endotracheal tube (ETT), or a supraglottic airway device such as a laryngeal mask airway (LMA), King Airway, or one of several other commercially available airway devices.

Endotracheal intubation is accomplished by inserting an airway device into the mouth, down through the throat and larynx, and into the trachea. This procedure creates an artificial passageway through which air can flow freely and continuously, in and out of a patient's lungs. Endotracheal intubation also prevents the patient's airway from collapsing or occluding.

It is very important that the airway device be placed and maintained in the correct position in the trachea. If the device moves out of its proper position in the trachea and into the right main stem bronchial tube, the left lung will not be ventilated properly leading to atelectasis and associated pulmonary complications, while the right lung will be over ventilated leading to tension pneumothorax. Moreover, if the airway device moves completely out of the trachea and into the pharynx, esophagus or completely outside the body, the patient will become hypoxic due to the lack of ventilation to the lungs, a condition which typically results in life-threatening brain injury or death within a matter of only a few minutes.

Even after an airway device has been positioned correctly, subsequent movement of the patient can lead to inadvertent movement of the device, as hereinabove described. An intubated patient may restlessly move about and may also attempt to forcibly remove an airway device, whether conscious or subconscious, particularly if the patient is uncomfortable or having difficulty breathing, which can lead to panic.

Unintentional movement of either a patient or an airway device is not uncommon, particularly when the patient is moved from an out-of-hospital setting such as from the scene of an accident to an emergency department of a hospital. Further, anytime an intubated patient is to be moved, for example, not only from an ambulance to a trauma facility, but also from one hospital to another hospital, from one area of the hospital to another area in the same hospital (imaging, laboratory, operating theater), or from a hospital to an outpatient rehabilitation facility, unintentional movement of an airway device is a risk.

Inadvertent movement of an airway device may also occur as a result of moving external ventilation equipment, such as a conventional mechanical ventilator or bag valve mask. Typically, the external ventilation equipment is connected to the external end of the device by an air conduit to establish air flow to and from the lungs. Inadvertent pulling on, or other excessive movement of the air conduit, may not only disconnect it from the airway device, but may also transfer movement to the airway device, thereby shifting it from its proper position and causing unplanned extubation.

Unplanned extubation (UE) is a hazardous and costly problem in humans, a problem which studies have established occurs at an unacceptably high rate. For example, Statistics published by the Society for Critical Care Medicine states that in 2017 there were 1.65 Million intubated, mechanically ventilated ICU patients in the United States (Medicine, S.f.C.C. Critical Care Statistics 2017). A review of the world-wide medical literature suggests that the world-wide rate of unplanned extubation averages approximately 7.31% of extubated patients. Lucas de Silva, Unplanned Endotracheal Extubation in the Intensive Care Unit: Systematic Review, Critical Appraisal, and Evidence-Based Recommendations. Anesth Analg 2012; 114:1003-14. Applying the world-wide average to the U.S. figure above, an estimated 120,000 patients in the United States alone experience an unplanned extubation each year. Such unplanned extubations are costly, not only for patients who experience increased rates of morbidity and mortality, but also for hospitals, physicians and insurance companies who incur the liability costs associated therewith. The annual intensive care unit (ICU) bed cost associated with unplanned extubations in the United States alone is estimated at $4.9 Billion, which includes imaging, pharmacy, and laboratory expenses. (Extrapolated using data from the Carlson study referenced below and the cost of long-term care according to the U.S. Department of Health and Human Services National Clearinghouse for long-term care information. See also S. K. Epstein, M. L. Nevins & J. Chung, Effect of Unplanned Extubation on Outcome of Mechanical Ventilation, Am. Journal of Respiratory and Critical Care Medicine, 161: 1912-1916 (2000) which discusses the increased likelihood of long-term care outcome). Moreover, it is known for jury damage awards in personal injury lawsuits arising from unplanned extubations to be in excess of $35 M.

One common approach for securing an airway device (typically, an endotracheal tube) is with adhesive tape. Umbilical tape may be used as an alternative. Both present the same challenges. The tape is tied around the patient's neck and then wrapped and tied around the smooth outside surface of the endotracheal tube itself. Arranged in this fashion, the tape is intended to anchor the endotracheal tube to the corner of the patient's mouth and prevent its unintentional movement. While the use of tape in this manner provides some benefit, the restraint available from the tape usually diminishes because the tape becomes covered and/or saturated with blood, saliva, or other bodily fluids. Consequently, the endotracheal tube may be readily moved from its preferred position in a patient's trachea. In spite of its widespread use, adhesive or surgical tape is woefully inadequate in providing protection against movement resulting from the application of multidirectional forces such as bending, torsional/rotational or substantial lateral forces to the device, forces which may exceed forty-three (43) pounds in magnitude. Moreover, the tape may be readily contaminated as a result of being in direct contact with a patient's face, oral cavity and bodily fluids and may spread bacteria, germs, and the like throughout an entire healthcare facility, thus increasing the risk of infection to patients and staff alike.

The results of two studies of the restraint capabilities of current devices and methods are set forth in Tables 1 and 2 below. Such devices and methods do not provide sufficient resistance to prevent unplanned extubation. Clinically significant movement is defined as longitudinal movement of the airway device in a direction towards or away from the patient's mouth to a point where the tip of the airway device has moved beyond the larynx or vocal cords. Typically, such movement in a human patient is in the range of five (5) to seven (7) centimeters.

### Restraint Capabilities of Current Devices and Methods in Human Applications

TABLE 1

| | Median | Min | Max |
|---|---|---|---|
| Thomas Tube Holder | 12.98 | 2.64 | 22.44 |
| Adhesive Tape | 19.58 | 3.96 | 39.6 |
| Non-Adhesive Tape | 7.48 | 2.42 | 27.72 |

Force to Extubate (7 cm movement) in Lbs.
Owens, et al. Resuscitation (2009)

TABLE 2

| | Median | Min | Max |
|---|---|---|---|
| Adhesive Tape (Lillehei) | 19.5 | 15 | 25 |
| Tube Tamer | 12.9 | 10 | 15 |
| Precision Medical | 8.6 | 7 | 10 |
| Biomedix Endogrip | 10.7 | 6 | 12 |
| Thomas Tube Holder | 37 | 28 | 43 |

Force to Extubate (2 cm movement) in Lbs.
Carlson, et al. Annals of Emergency Medicine 2007

U.S. Pat. No. 8,001,969 issued on Aug. 23, 2011, and U.S. Pat. No. 8,739,795 issued on Jun. 3, 2014, both to Arthur Kanowitz, the inventor of the present invention, disclose airway stabilization systems which address many of the problems set forth above. Continuing research into ways of providing even more advanced and rapidly deployable airway stabilization systems have resulted in yet further improvements to the overall design of airway stabilization system components by adapting them to accommodate the wide range of ETT tube sizes and patient facial geometries in both adult and pediatric patients.

Environments exist, however, wherein a patient is stationary and exposed to little or no movement, and the likelihood of the application of longitudinal, transverse, or rotational forces to a ventilation device positioned in a patient's airway of such magnitude as to result in unintentional and clinically significant movement thereof is low. By way of example, a patient undergoing a surgical procedure in an operating room or who has been moved post-op to a recovery room is in a stable environment wherein less concern for dislodgement of an airway device exists. Typically, in the operating room, the clinician providing anesthesia is at the head of the bed and attentive to the airway. This allows the clinician to intervene quickly to prevent extubation of the ETT. The robust stabilization systems which find application in emergency field situations, patient transport, and other high-risk scenarios such as the ICU, may not be necessary and are typically not acceptable to most clinicians providing anesthesia. In general, adhesive tape is the standard of care in the operating room.

In view of the foregoing, it will be apparent to those skilled in the art from this disclosure that a need exists for an uncomplicated airway securement apparatus which is easy to both install on and remove from a patient in a low-risk stable environment while at the same time is sufficiently robust to maintain the airway device in its preferred position in a patient's trachea and to prevent clinically significant movement thereof. A preferred apparatus would reduce the risk of the spread of infection throughout a medical facility by eliminating surgical tape used by prior art techniques to secure airway devices in stable environments that is contaminated by its exposure to a patient's body fluids. The method by which tape can spread infection throughout the hospital is that a provider may use tape on one patient, but not use the entire roll. The provider then carries that tape in a pocket and may use it on another patient. It should be noted that this practice is less likely to occur in an operating room setting. Nonetheless, it is a risk that can be reduced via the use of an improved airway securement apparatus such as disclosed herein. Optimally, the securement apparatus should be simple and economical in design and possess the capability of being used to secure airway devices of different sizes within a given range in patients having varying facial and anatomical geometries. The present invention addresses these needs in the art as well as other needs, all of which will become apparent to those skilled in the art from the accompanying disclosure.

### SUMMARY OF THE INVENTION

To address the aforementioned needs in the art, an airway securement apparatus is provided that is adapted for use in low risk and stabile environments, by way of example and not of limitation, operating and recovery rooms in hospitals and health care facilities, and similar environments where a patient is stationary and exposed to little or no movement and wherein the likelihood of the application of forces to the patient and/or the airway apparatus of a magnitude sufficient to cause clinically significant movement of the airway device is low.

The airway securement apparatus may be easily, efficiently and quickly fitted to and removed from any airway device within a range of sizes that may be used with human patients to maintain an airway in a patient's trachea to a patient's lungs, the patient having a head, a face, a chin, a nose, a mouth, an oral cavity, vocal cords or larynx, a thoracic area, a chest, a trachea having a length and forming an airway in the patient, and a carina defining a point at which the trachea separates into a left and a right bronchial tube. The securement apparatus prevents clinically significant movement of the airway device with respect to a patient's vocal cords in response to the application of forces in any direction to the device, be they longitudinal, torsional/rotational or bending.

The airway device has a flexible elongate body which conforms to a patient's trachea after it is installed in the patient. The airway device includes a continuous sidewall having outer and inner surfaces extending between a proximal (patient-end) and a distal (machine-end) portion thereof, thereby forming a hollow conduit or body through which the airway is established.

In an embodiment, a securement apparatus includes a base or support member secured to the patient and a tower structure, also referred to herein as simply “the tower,” operatively connected thereto or integrally formed therewith, the elements and operation of which are described in greater detail below. In an embodiment, the base and the tower are formed of the same material, typically a synthetic and preferably silicone. However, it is to be understood that other embodiments may include a base and a tower operatively connected to one another and formed of dissimilar materials respectively without departing from the scope of the present invention. Moreover, other materials having properties suitable for the application may be used without departing from the scope of the present invention.

The securement apparatus is configured to interact in securing engagement with the continuous sidewall of the airway device to prevent clinically significant movement of the patient end of the airway device with respect to the patient's vocal cords. In the operating room, the securement apparatus will be placed on the patient and then left in place until the procedure is over. It is not typically removed and placed back on. With the apparatus of the instant invention, even if a patient is proned, the device would be placed and left in place until the procedure is completed. However, proning is a higher risk procedure and therefore the additional use of a strap would be warranted in that situation.

The base or support member is of unitary construction to allow greater ease of application and installation on and/or removal of an airway device from a patient.

The tower extends outwardly from the base along a longitudinal axis in a direction away from a patient's face. The tower includes a continuous cylindrically-shaped body extending circumferentially about and along the longitudinal axis, the body having a first or bottom end operatively connected to the base and second or top end, an inner surface and an outer surface, each of the surfaces extending intermediate the first and second ends, and an elongate aperture or slot formed in the body intermediate the inner and outer surfaces and extending between the first and second ends in a direction parallel to the longitudinal axis. The aperture or slot is adapted to be pried open to fit over the flexible elongate body portions of multiple sizes of airway devices.

In an embodiment, the outer surface of the tower includes a recessed portion adapted to guide the application of a locking device to the tower, as described below.

In another embodiment, the second edge is beveled and includes a rounded fillet.

The base includes a rectangularly shaped body extending along a longitudinal axis, the body having a thickness, first and second ends, first and second sides, an upper surface and a lower surface, the upper and lower surfaces extending parallel to one-another intermediate the ends and the sides, and at least one slot or aperture formed in each end and adapted to receive a securement apparatus.

In an embodiment, the securement apparatus has one or more strips of tape, the tape having an upper or top surface and a bottom or lower surface, the lower surface having an adhesive material thereon adapted to removably secure each of the one or more tape strips to a patient's face.

In another embodiment, the base has a width, the width being sized to cover the upper lip of a patient.

In yet another embodiment, the upper surface of the base includes a channel adapted to receive a securing band, strap or a tape strip.

In still another embodiment, the lower surface of the base includes a section of adhesive secured thereto, the adhesive having removable backing to protect it prior to installation on a patient, so that the base may be removably adhered to a patient's upper lip to increase the stability of the securement apparatus after installation on a patient and is such that when the use of the device is complete, it can be removed from the patient's skin, i.e. the bond is not permanent to the skin.

In yet another embodiment, the airway securement apparatus includes an extended lower end portion which forms a bite block adapted to be inserted in a patient's oral cavity to prevent occlusion of an airway device and to protect an airway device from damage arising from a patient's biting the airway device or clenching his or her teeth.

In still another embodiment, the tower comprises silicone and the airway device comprises a plastic material.

In yet another embodiment, the airway securement apparatus utilizes the interaction of the silicone of an inner surface of the tower with the plastic material of the airway device to retain the airway device in secure engagement with the tower.

In an embodiment, the airway securement apparatus further includes a locking device comprising a section of tape wrapped circumferentially around the tower whereby the tower and tube interface is maintained in touching engagement and thus preventing the tower from opening when pressure is applied thereto.

If a patient must be transferred to another treatment unit in a hospital or surgical center such as an intensive care unit (ICU) and a more robust airway securement apparatus is needed due to an increased risk for UE during movement of the patient. In an embodiment, a method for maintaining the precise, safe and effective intubation and ventilation of a patient during the transition is disclosed. An interlock collar may be placed on the second or top end of the tower for correct positioning and secured to the continuous sidewall of the airway device. The collar includes a pair of pivotally interconnected elongate c-shaped cylindrical members extending outwardly from the patient's face coaxially with the longitudinal axis of the airway device or tower. Each of the elongate cylindrical members includes first and second ends and a body portion having an inner surface and an outer surface extending therebetween. The outer surface of at least one of the pair of cylindrical members includes at least one annular flange and structural recess extending radially outwardly from the outer surface. and adapted to operatively interact with one of a plurality of structural recesses formed intermediate a pair of substantially uniformly spaced-apart annular flanges positioned axially along an inner surface of at least one of a pair of pivotally interconnected clamshells adapted to be releasably closed about the collar to retain the airway device in a preselected position in a patient's airway, as described in greater detail below. The inner surface of at least one of the c-shaped cylindrical members may be coated with an adhesive material, by way of example and not of limitation, a pressure sensitive adhesive (PSA) adapted to adhesively engage the outer surface of an airway device.

In yet another embodiment, the inner surface of the interlock collar may be textured, for example, like the surface texturing found on a porcupine quill, minute suction structures, or micro texture surface technologies such as a Sharklet® micropattern to selectively prevent axial motion along the B-B axis of an airway device in one or both axial directions.

In still another embodiment, the interlock collar includes a mechanism for selectively deploying a bonding agent such as cyclohexanone intermediate the inner surface of at least one of the pair of pivotally interconnected elongate c-shaped cylindrical members and the outer surface of the continuous sidewall of an airway device.

In another embodiment, at least one of the c-shaped cylindrical members of the interlock collar includes a vertical flex beam member adapted to releasably engage airway devices having different diameters.

In an embodiment, the interlock collar includes a latch mechanism adapted to secure the interlock collar in a selected position on an airway device.

After the interlock collar is placed on the second or top end of the tower for correct positioning and secured to the continuous sidewall of the airway device as noted above, the airway securement apparatus that is adapted for use in low risk and stabile environments is removed, and a more robust airway securing and stabilizing system is installed on the patient and operatively connected to the collar. In an embodiment, the robust airway securing system includes a frame, bridge or support member secured to the patient and a tower structure or clamshell-type clamping member operatively connected thereto. The clamping member is configured to interact in clamping engagement with the continuous sidewall of the airway device via the collar previously positioned on the second or top end of the tower for securement to the continuous sidewall of the airway at the proper location.

In this embodiment, the clamping member includes a pair of oppositely disposed pivotally interconnected c-shaped collars or clamshells, each collar or clamshell having a first end and a second end and a body portion extending therebetween, the body portion having an inner surface and an outer surface, the inner surface of at least one of the body portions including a plurality of substantially uniformly spaced-apart annular flanges positioned axially along the inner surface of the body portion and extending substantially inwardly therefrom, and a plurality of structural recesses positioned axially along the inner surface of the body portion intermediate an adjacent two of the plurality of substantially uniformly spaced-apart annular flanges the ribs and structural recesses of the clamshells. The at least one annular flange and structural recess extending radially outwardly from the outer surface of at least one of the collar's cylindrical members is adapted to operatively interact with one of a plurality of structural recesses formed intermediate the substantially uniformly spaced-apart annular flanges positioned axially along the inner surface of the at least one of the clamshells to retain the airway device in a preselected position in a patient's airway. Thus protected, the patient may be moved to another unit or an ICU for subsequent treatment and/or recovery.

These and other features, aspects and advantages of the present invention will become apparent to those skilled in the art from the following detailed description of preferred embodiments taken in connection with the accompanying drawings, which are briefly summarized below, and by reference to the appended claims.

### BRIEF DESCRIPTION OF THE DRAWINGS

Referring now to the attached drawings which form a part of this original disclosure:

FIG. 1 is a front bottom right perspective view of portions of an airway securement apparatus for application in stable and secure environments that minimize the risk of unplanned extubation, in accordance with an embodiment of the present invention;

FIG. 2 is a top front perspective view of the airway securement apparatus of FIG. 1;

FIG. 3 is a bottom elevation view of the airway securement apparatus of FIGS. 1 and 2;

FIG. 4 is a top plan view of the airway securement apparatus of FIGS. 1-3;

FIG. 5 is an enlarged right side elevation view of the airway securement apparatus of FIGS. 1-4;

FIG. 6 is a front bottom right perspective view of an airway securement apparatus which includes an integral bite block adapted to protect an airway device from crushing and other damage arising from a patient's biting or teeth clenching;

FIG. 7 is a right side elevation view of the airway securement apparatus of FIG. 6;

FIG. 8 is a front plan view of an airway securement apparatus for application in stable and secure environments shown installed on a patient;

FIG. 9 is a right side perspective view of an airway securement and stabilization system for use in situations where a robust system is required to prevent unplanned extubation of a patient in accordance with another embodiment;

FIG. 10 is a side elevation view of an interlock collar of the system of FIG. 9 shown in an open configuration positioned on an airway device in accordance with an embodiment;

FIG. 11 is a side elevation view of the interlock collar of FIG. 10 shown in a closed configuration on an airway device;

FIG. 12 is a front bottom left perspective view of portions of the airway securement apparatus for application in stable and secure environments of FIG. 1 illustrating the placement of an interlock collar thereon;

FIG. 13 is a top side elevation view of the airway securement apparatus and interlock collar of FIG. 12;

FIG. 14 is a right side perspective view of the airway securement apparatus and interlock collar of FIGS. 12 and 13;

FIG. 15 is a top plan view of the airway securement apparatus and interlock collar of FIGS. 12-14 illustrating the interlock collar enclosed about an airway device;

FIG. 16 is a side perspective transparent view of an airway device installed in a patient;

FIG. 17 is a bottom perspective transparent view of an airway device installed in a patient and viewed from the patient's chin illustrating an airway securement apparatus for application in stable and secure environments positioned for installation on the patient;

FIG. 18 is a bottom perspective transparent view of the patient and the airway securement apparatus for application in stable and secure environments illustrating the removal of a protective backing layer from an adhesive material secured to the bottom of the base;

FIG. 19 is an oblique perspective view of the patient and the airway securement apparatus of FIG. 18 illustrating the airway securement apparatus positioned on an upper lip of a patient;

FIG. 20 is an oblique perspective view of the patient and the airway securement apparatus of FIG. 19 illustrating a tower portion of the airway securement apparatus opened in preparation for receiving the airway device;

FIG. 21 is an oblique perspective view of the patient and the airway securement apparatus of FIG. 20 showing the airway device positioned in the tower;

FIG. 22 is an oblique perspective view of the patient and the airway securement apparatus of FIG. 21 showing the airway device positioned in the tower and having a securement apparatus extending circumferentially around the tower and the airway device;

FIG. 23 is an oblique perspective view of the patient and the airway stabilization apparatus of FIG. 22 showing the removal of a protective backing layer from a plurality of securement straps in preparation for placing the straps on a patient's face;

FIG. 24 is an oblique perspective view of the patient and the airway securement apparatus of FIG. 23 showing the positioning of the plurality of securement straps secured to a patient's face;

FIG. 25 is an oblique perspective view of the patient and the airway securement apparatus of FIG. 24 showing the application of an additional safety strap for securing the airway securement apparatus to a patient;

FIG. 26 is an oblique perspective view of the patient and the airway securement apparatus of FIGS. 17-25 showing an interlock collar in an open configuration prepared and positioned for placement on the top of the tower portion of the airway securement apparatus;

FIG. 27 is an oblique perspective view of the patient and the airway securement apparatus of FIG. 26 showing a protective backing film being removed from a layer of pressure sensitive adhesive (PSA) on an inner face of the interlock collar;

FIG. 28 is an oblique perspective view of the patient and the airway securement apparatus of FIGS. 17-27 showing an interlock collar in an open configuration positioned on the top of the tower portion of the airway securement apparatus;

FIG. 29 is an oblique perspective view of the patient and the airway securement apparatus of FIG. 28 showing an interlock collar in a closed configuration about an airway device and positioned on the top of the tower portion of the airway securement apparatus;

FIG. 30 is an oblique perspective view of the patient and the airway securement apparatus of FIG. 29 showing the safety strap removed and the plurality of securement straps detached from the patient's face in preparation for removal of the airway securement apparatus from the patient;

FIG. 31 is an oblique perspective view of the patient and the airway securement apparatus of FIG. 30 illustrating the tower portion of the airway securement apparatus opened and the airway device removed therefrom in preparation for removing the airway securement apparatus from the patient;

FIG. 32 is an oblique perspective view of the patient and an airway device and an interlock collar installed in a patient illustrating the airway securement apparatus of FIG. 31 removed from the patient;

FIG. 33 is a bottom perspective transparent view viewed from the chin of the patient and an airway device and an interlock collar installed in a patient illustrating the airway securement and stabilization system of the embodiment of FIG. 9 positioned for installation on the patient;

FIG. 34 is a bottom perspective transparent view viewed from the chin of the patient and an airway device and an interlock collar installed in a patient showing a protective backing film being removed from a layer of pressure sensitive adhesive (PSA) on an inner face of a pair of cushions or pads operatively connected to the airway securement and stabilization system embodiment of FIG. 9;

FIG. 35 is an oblique perspective transparent view of the patient and an airway device and an interlock collar as shown in FIG. 34 illustrating the airway securement and stabilization system of the embodiment of FIG. 9 installed on the patient;

FIG. 36 is an oblique perspective transparent view of the patient and an airway device and an interlock collar as shown in FIG. 35 illustrating the airway device positioned in the airway securement and stabilization system of the embodiment of FIG. 9;

FIG. 37 is an oblique perspective transparent view of the patient and an airway device and an interlock collar as shown in FIGS. 35 and 36 illustrating the interlock collar in a closed configuration on the flexible elongate body portion of the airway device;

FIG. 38 is an oblique perspective transparent view of the patient, an airway device and an interlock collar as shown in FIGS. 35-37 illustrating the positioning of a plurality of securement straps operatively connected to the airway securement and stabilization system and adapted to securing it to a patient's face;

FIG. 39 is a front top right side perspective view of portions of an airway securement apparatus for application in stable environments in accordance with another embodiment of the present invention;

FIG. 40 is a rear right side perspective view of the airway securement apparatus of the embodiment of FIG. 39;

FIG. 41 is a bottom side elevation view of the airway securement apparatus of the embodiment of FIGS. 39. and 40 illustrating a hydrocolloid layer covered by an adhesive layer operatively connected to a bottom surface thereof and covered by a protective liner;

FIG. 42 is a bottom side elevation view of the airway securement apparatus of the embodiment of FIGS. 39-41 illustrating the step of removing the protective liner from the adhesive layer; and

FIG. 43 is an oblique perspective transparent view of the patient illustrating the airway securement and stabilization system of the embodiment of FIG. 9 installed on the patient and having an interlock collar positioned in a securing apparatus portion of the system and an airway device being positioned therein in a process of transitioning from a stable environment to an environment where a more robust securement and stabilization system is required.

### DETAILED DESCRIPTION OF THE PREFERRED EMBODIMENTS

Selected embodiments of the present invention will now be explained with reference to the drawings. It will be apparent to those skilled in the art from this disclosure that the following descriptions of the embodiments of the present invention are provided for illustration only and not for the purpose of limiting the invention as defined by the appended claims and their equivalents.

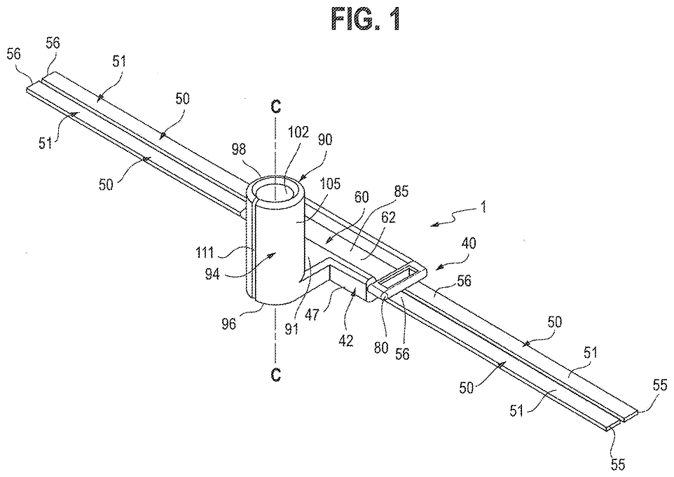

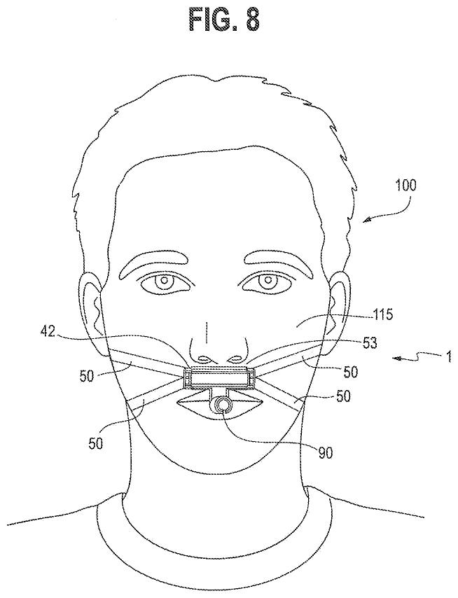

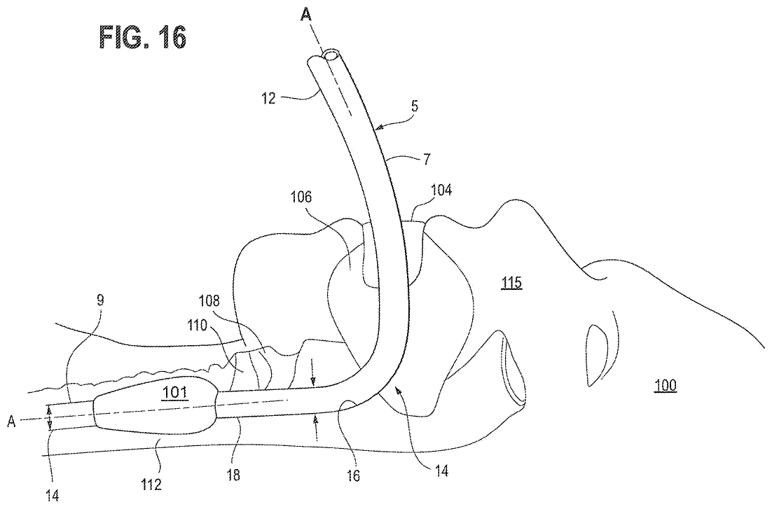

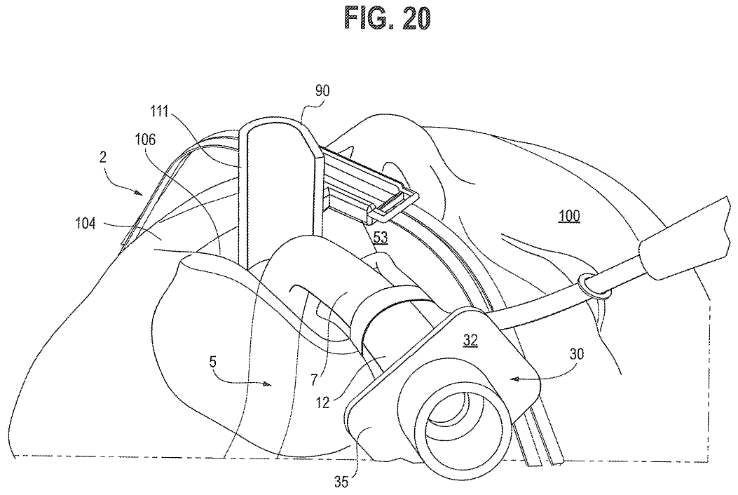

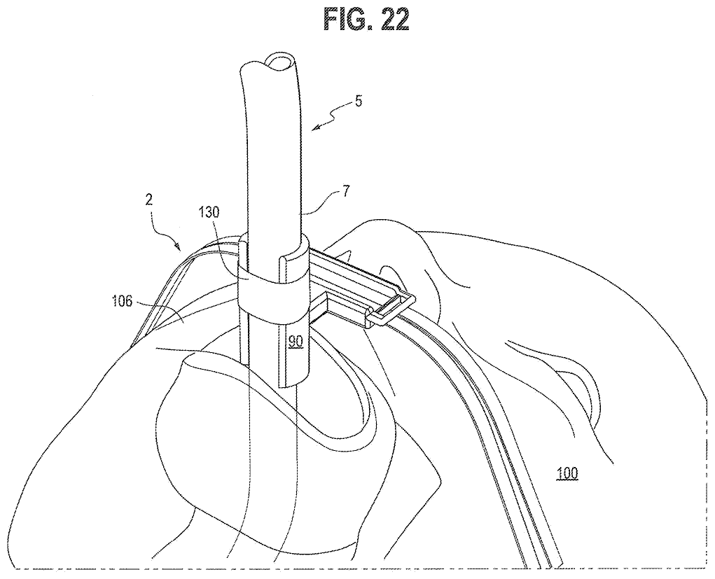

Referring initially to FIG. 1, portions of an airway securement apparatus for use in stable environments shown generally at numeral 1 are illustrated in accordance with an embodiment of the present invention. The airway securement apparatus, referred to herein as “the apparatus” for purposes of brevity, is used to maintain an airway in a patient shown at 100 in FIG. 8. As best shown in FIGS. 16 and 22, the apparatus includes an airway device depicted generally at 5 which has a flexible elongate body 7 extending along a longitudinal axis A-A and having a length, an internal diameter S₁ and an external diameter S₂, a patient-end portion 9, a machine end portion 12 and a continuous sidewall 14 having an internal surface 16 and an external surface 18 extending between the patient and the machine end portions, thereby forming an airway. As illustrated in FIG. 20, a connector assembly 30 is operatively connected to the machine end 12 of the elongate body of the airway device and is adapted to connect a respiratory connector, also referred to as a 15 mm connector, 32 to the airway device. The connector also includes “wings” 35 that are grips for inserting the connector onto or removing it from the endotracheal tube.

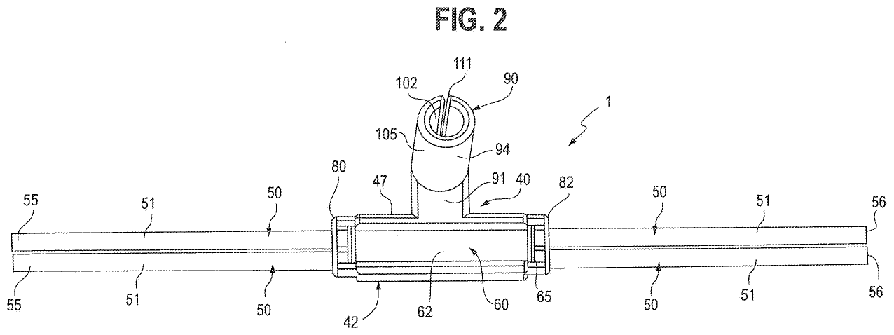

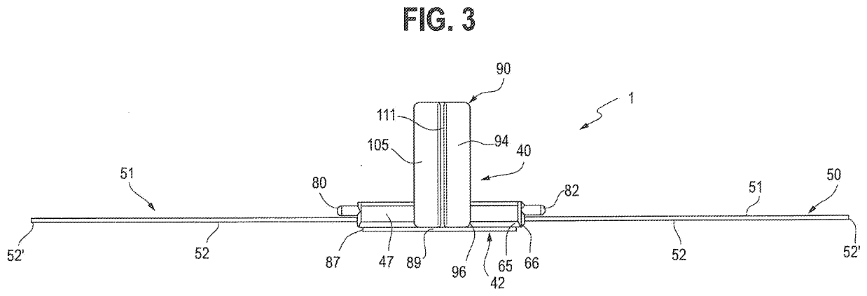

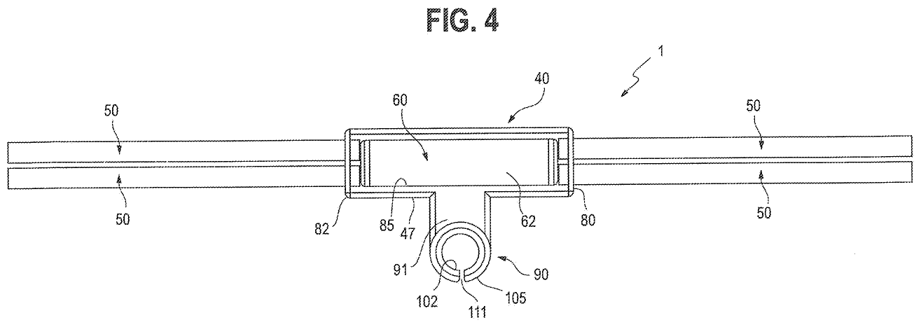

Referring back to FIGS. 1-7, the apparatus 1 includes a securing apparatus shown at 40, the airway device and securing apparatus cooperating to ventilate a patient 100 (FIGS. 8 and 16) by maintaining an air passageway or airway to a patient's lungs via the patient's mouth 104, oral cavity 106, throat 108, past a patient's vocal cords or larynx 110 into a patient's trachea 112 and to a patient's carina (the point where the trachea bifurcates into the bronchial tubes) for respiration of the patient. By way of example and not of limitation, the airway device may be in the form of an endotracheal tube (ETT) as shown in the accompanying figures, one of several commercially available endotracheal tubes or one of several commercially available supraglottic airway devices such as a King LT™ airway device manufactured by King Systems, Noblesville, Indiana, or a laryngeal mask airway (LMA) such as a LMA Classic™ manufactured by LMA North America, San Diego, California. Airway devices are typically made of a suitable flexible plastic material, as is known in the art.

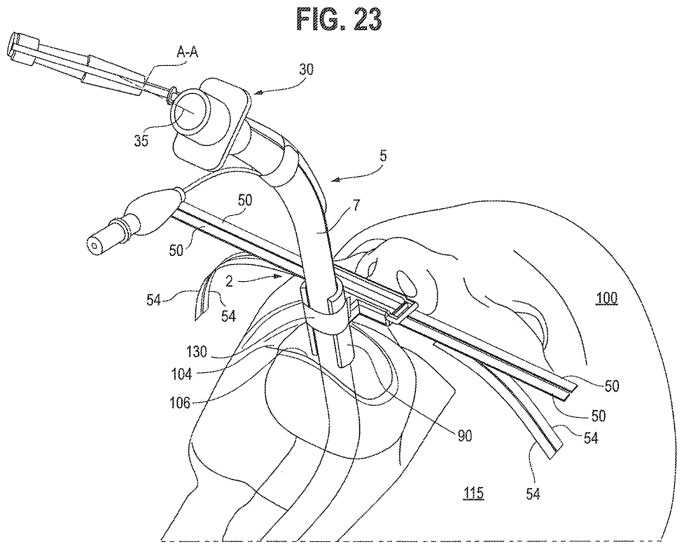

The securing apparatus 40 includes a base 42 adapted to be secured to a patient and a collar or tower structure 90, also referred to herein as simply “the tower”, operatively connected thereto or integrally formed therewith, the elements and operation of which are described in greater detail below. The base and the tower are formed of the same material, typically a synthetic and preferably silicone; however, it is to be understood that other materials having similar properties may be used without departing from the scope of the present invention. The base may be secured to a patient 100 by a suitable securement device or attachment mechanism, by way of example and not of limitation, one or more continuous flexible tape strips 50 adapted to be removably affixed or adhered to a patient's face 115 (FIG. 8). Each of the one or more tape strips includes an upper surface 51, a lower surface 52, first and second ends 55 and 56, and a midsection 60. The midsection 60 has an upper surface 62 and a lower surface 65, the lower surface being adhered to the base via a layer of adhesive material 66 operatively connected thereto and suitable for use in medical applications. The lower surface 52 of each of the one or more tape strips includes an adhesive layer 52′ protected by a removable backing strip 54 (FIG. 23), the adhesive layer being of a non-irritating formulation which may be safely affixed to a patient's facial skin. As shown in FIG. 8, the tape strips 50 are adhered to the patient's face 115 in an x-pattern which lessens the pressure on the patient's face, thereby reducing the risk of pressure sores and decreased blood flow thereto.

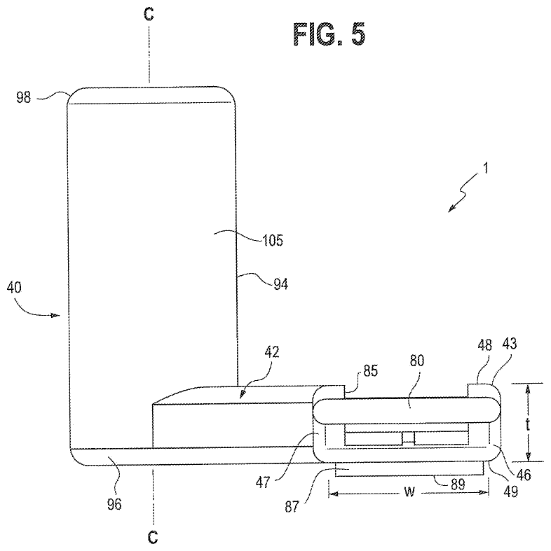

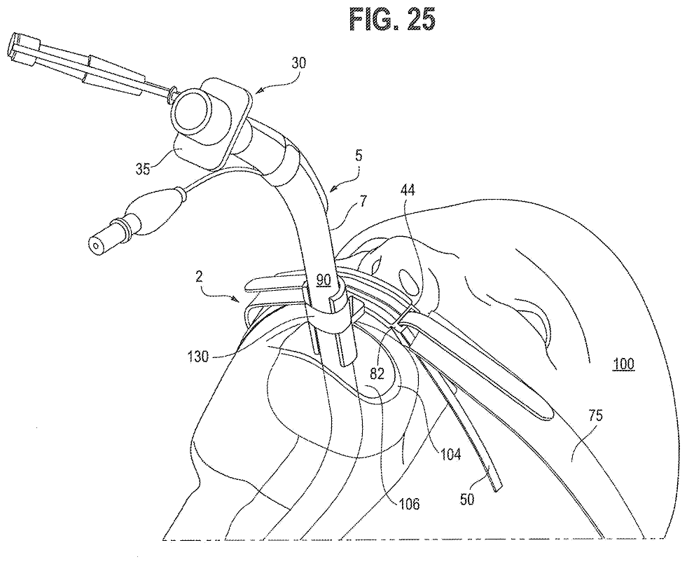

Referring to enlarged FIGS. 5-7, the elements of the base 42 are shown in greater detail. The base 42 includes a rectangularly shaped body 43 extending along a longitudinal axis B-B, the body having a thickness t, first and second ends 44, 45, first and second sides 46, 47, an upper surface 48 and a lower surface 49, the upper and lower surfaces extending parallel to axis B-B and to one-another intermediate the ends and the sides. Each end 44, 45 includes a cross member 80, 82 respectively secured thereto and extending intermediate the first and second sides 46, 47, each cross member being adapted to be connected to a securement device, for example the straps 75 shown in FIGS. 25-29. The base 43 further includes a channel 85 formed in the upper surface thereof intermediate the first and second ends 44, 45 and first and second sides 46, 47, the channel being adapted to receive midsection 60, which is adhesively bonded thereto via the adhesive material 66 located on the lower surface 65.

As best viewed in FIGS. 5 and 8, the width w of the base is sized to fit on an upper lip 53 of a patient. An adhesive layer 87 having a removable overlaying backing or protective layer or film 89 (FIG. 5) positioned thereon is secured to the lower surface of the base. Prior to placement of the apparatus on a patient, the backing layer is removed, and the base is releasably positioned on and adhered to the patient's upper lip 53 for enhanced stability. The adhesive is formulated to have no skin irritating properties.

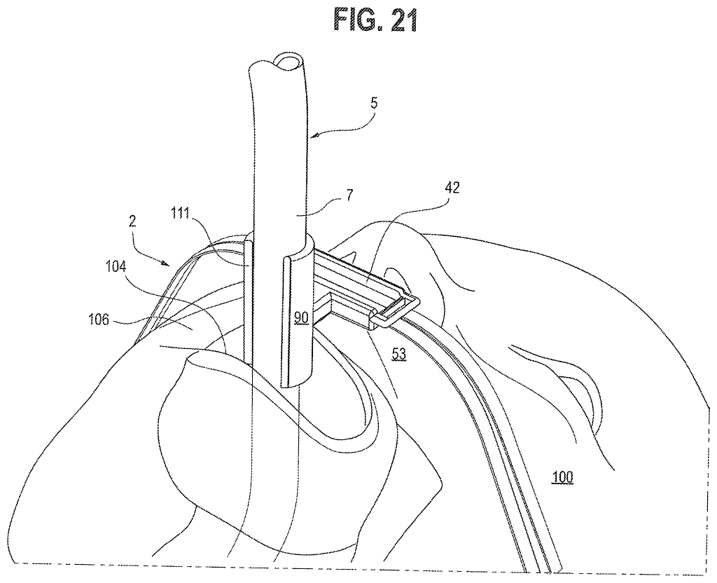

Referring again to FIGS. 1-4 and also to the enlarged FIG. 5, the securing apparatus 40 includes a cylindrically shaped collar or tower structure 90 operatively connected to or formed integrally with the base 42 via a connecting member 91 extending laterally outwardly from side 47 of the base 42. In one embodiment, the base and the tower are formed of the same material, typically a synthetic and preferably silicone; however, it is to be understood that other materials having properties similar to the properties of synthetic materials or silicone may be used without departing from the scope of the present invention Moreover, the base and the tower may each be formed of a different material which possesses properties suitable for the intended application, the base and tower being operatively connected via adhesive bonding, threaded fasteners and the like. The tower extends outwardly from the base along a longitudinal axis C-C which extends coaxially with the longitudinal axis A-A of the airway device in a direction away from a patient's face (FIG. 21). The tower includes a continuous cylindrically-shaped body 94 extending circumferentially about and along the longitudinal axis, the body having a first or bottom end 96 operatively connected to the base and second or top end 98, an inner surface 102 and an outer surface 105, each of the surfaces extending intermediate the top and bottom ends, and an elongate aperture or slot 111 formed in the body intermediate the inner and outer surfaces and extending between the top and bottom ends in a direction parallel to the longitudinal axis C-C. The aperture or slot is adapted to be pried open to fit over the flexible elongate body portions of multiple sizes of airway devices. The second or top end edge is beveled and includes a rounded fillet 112.

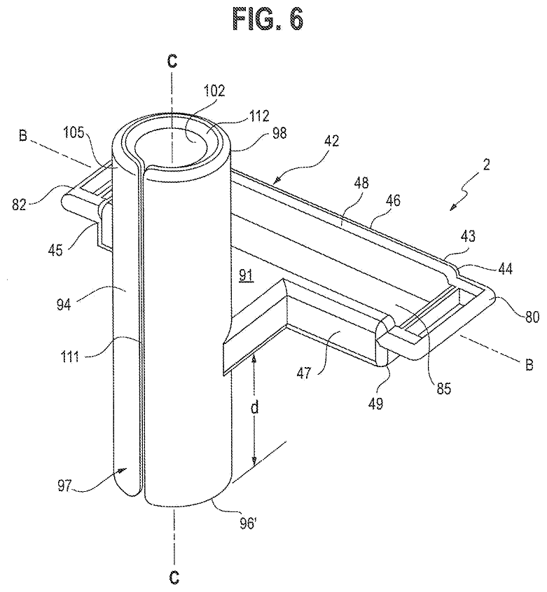

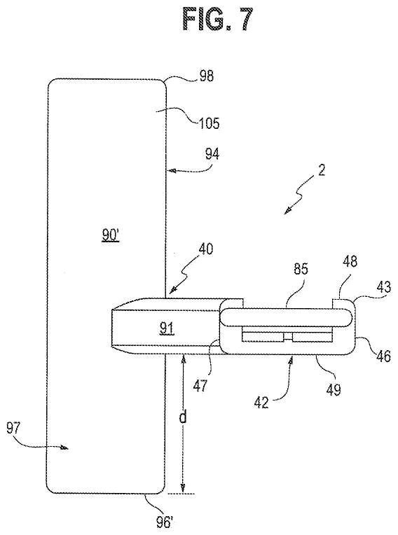

Referring to FIGS. 6 and 7, an airway securement apparatus 2 is illustrated that is identical to the airway securement apparatus 1 but in which a generally cylindrically shaped tower structure 90′ includes a bottom end 96′ that extends a distance d from the bottom of the connecting member 91 in a direction toward a patient when the apparatus is installed. The extended bottom end portion forms a bite block 97 which, when inserted in a patient's oral cavity, protects an airway device from crushing and other structural damage to the airway device that might result from a patient's biting the tubular body of the airway device or from clenching his or her teeth.

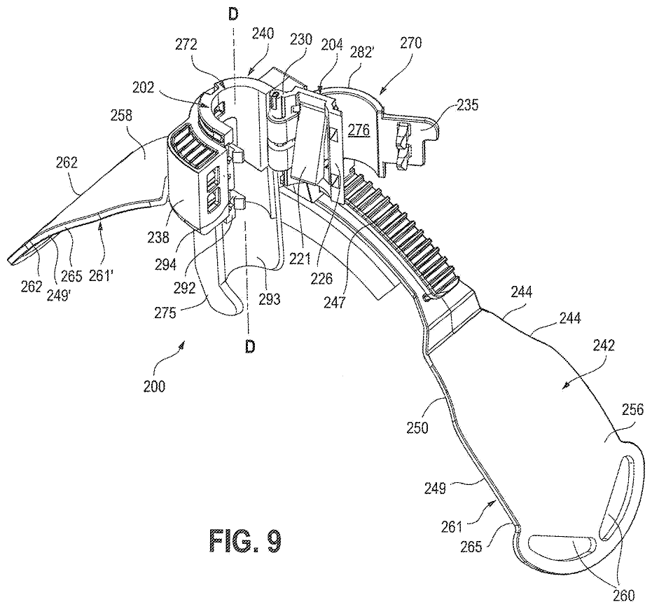

As will be described in greater detail below with respect to the installation methods of the present invention, if a patient must be transferred to another treatment unit in a hospital or surgical center such as an intensive care unit (ICU) and a more robust airway securement and stabilization system is needed for this high risk movement, an airway securement and stabilization system such as the system 200 shown in FIG. 9 may be exchanged for the apparatus of FIG. 1 et seq. without having to remove an installed airway device from a patient.

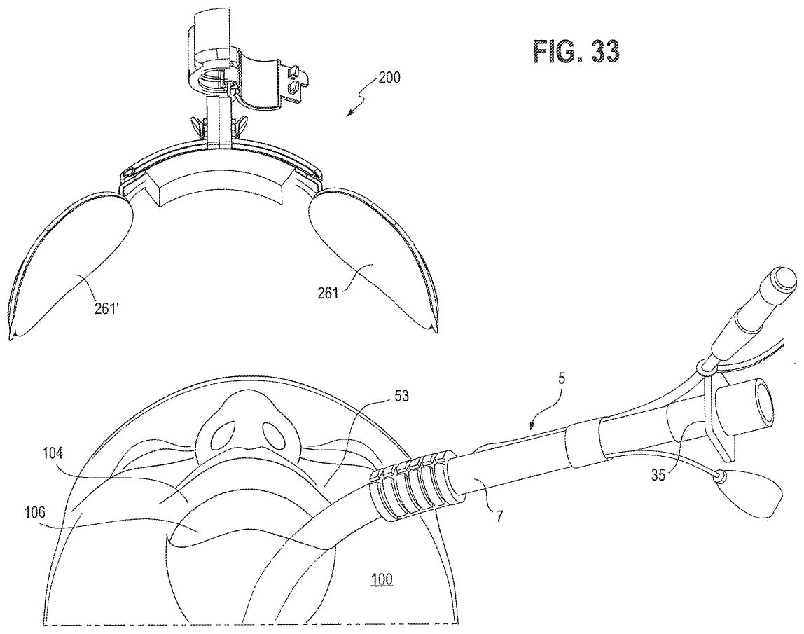

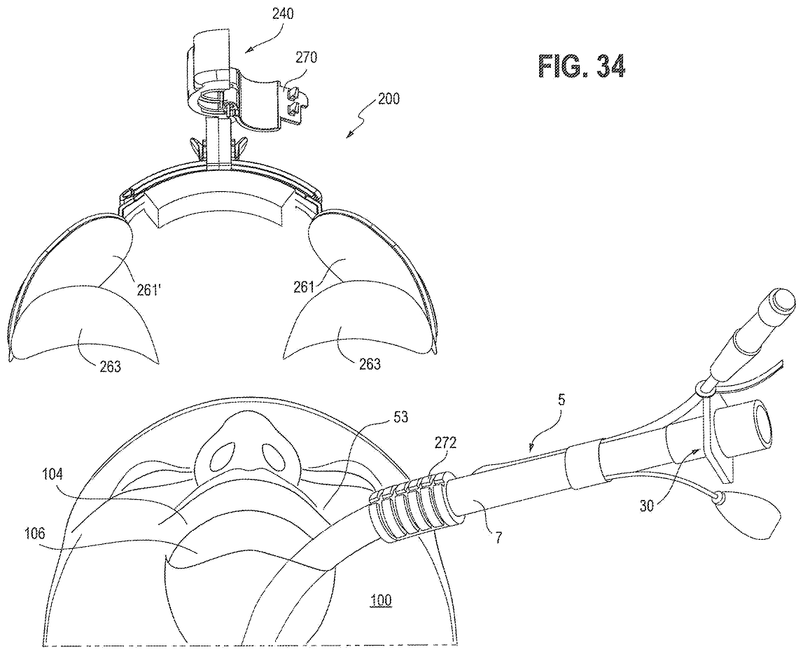

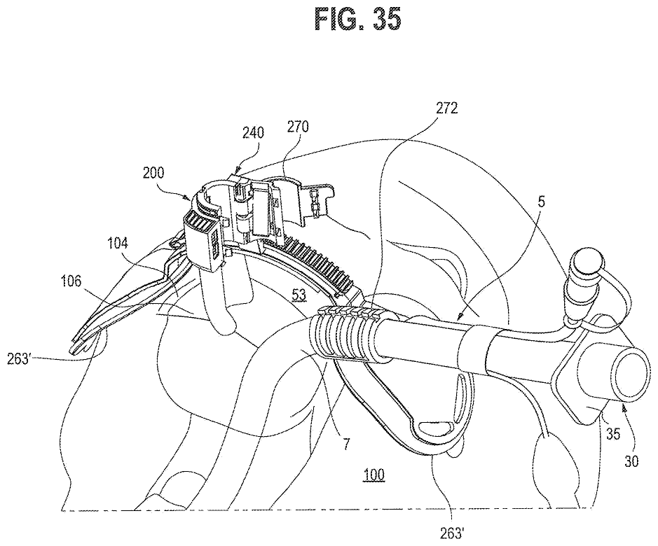

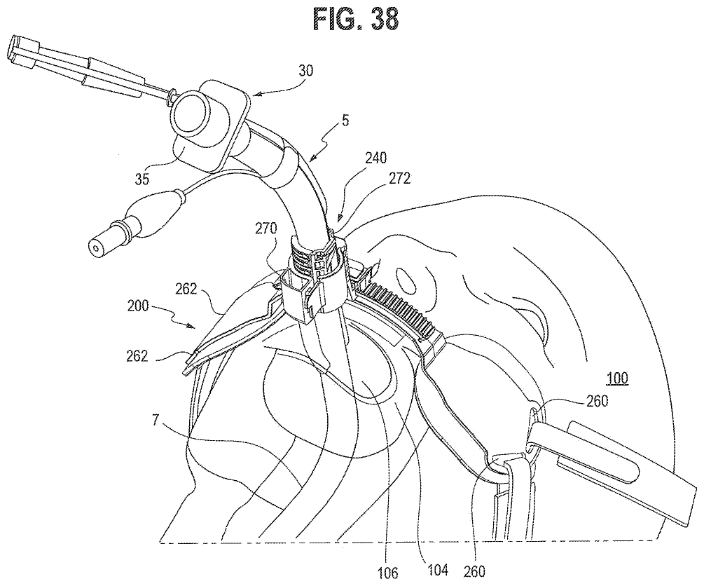

Referring now to FIG. 9, the airway securement and stabilization system 200 includes a securing apparatus 240 and a supporting bridge or frame 242 which may be secured to a patient's face by a suitable attachment apparatus, by way of example and not of limitation, one or more straps, for example straps 75 (FIG. 25) extending around the patient's head and securable by buckles, self-fastener, hook and loop closures or other suitable attachments, as is known in the art. The bridge or frame is preferably of unitary construction and in a generally symmetrical configuration contoured to permit it to conform to a patient's face when it is secured in position. It may be formed of plastic, rubber, metal, composite material, or other suitable materials having the desired physical properties for the application. The frame includes a body 244, an upper or outer surface 247 facing away from the patient and a lower or bottom surface 249 facing toward the patient, the upper and lower surfaces being interconnected by a pair of oppositely disposed, spaced apart side portions 250, 250′ and first and second oppositely disposed end portions 256, 258 respectively, each end being shaped to have a cheekpad secured thereto, as will be described in greater detail below with respect to FIGS. 33-38. Each end portion 256, 258 further includes one or more apertures or slots 260, 262 formed therein respectively and adapted to receive the attachment apparatus or strap 75. The frame may also include a cheek pad or layer 261, 261′ of cushioning material covered by a protective backing 263 (FIG. 34) releasably secured to a lower surface 249, 249′ of each respective end to reduce pressure on a patient's face and to provide additional comfort for the patient. By way of example and not of limitation, the cushioning material may be a hydrocolloid absorbent, waterproof material, or a silicone gel pad for use when a patient having burns, skin tears, rashes, facial hair where an adhesive would be contraindicated. The cushioning material is operatively secured to the lower surfaces 249, 249′ by a suitable securing material such as a releasable adhesive or bonding agent or a hook and loop closure or self-fastened fastener to allow changeout or replacement thereof when the cheek pads become soiled without being bulky or uncomfortable. As best shown if FIGS. 35-37, in an embodiment, a skin contact adhesive 263′ is applied to the cushioning material that will have a perforated pattern to allow moisture to wick to the underlying absorbent hydrocolloid material, thus permitting multi-day wear of the airway stabilization system.

The securing apparatus 240 of the stabilization system 200 includes a generally cylindrically-shaped tower structure or clamshell-type clamping member 270 operatively connected to the frame 242. The tower structure is configured to interact in clamping engagement with the continuous sidewall 14 of the airway device 5 via an adaptable airway securement device or interlock collar 272 which is removably and adjustably positioned in and adjustably connected to the securing apparatus via the tower structure and cooperates therewith to prevent clinically significant movement of the distal end 9 of the airway device with respect to the vocal cords of the patient. The tower structure/clamping member 270 and the collar 272 extend in a substantially perpendicular direction from the outer surface 247 of the frame 242 coaxially along axis D-D. The clamping member 270 includes a pair of oppositely disposed pivotally interconnected c-shaped clamshells (specifically, clamshell clamping member 275 and door member 276). The clamshells are pivotally interconnected, for example, by a pin 230 as shown in FIG. 9.

An inner surface of clamshell 275 of the clamshell clamping member 270 includes a plurality of substantially uniformly spaced-apart annular flanges 292 positioned axially along the inner surface thereof and extending substantially inwardly therefrom, and a plurality of structural recesses 294 positioned axially along the inner surface of the collar or clamshell intermediate an adjacent two of the plurality of substantially uniformly spaced-apart annular flanges. In an embodiment, the tower structure may include a bite block 293 operatively connected thereto and adapted to be positioned in a patient's oral cavity to prevent crushing of the airway device and potentially extremely dangerous disruption of ventilatory air to the patient.

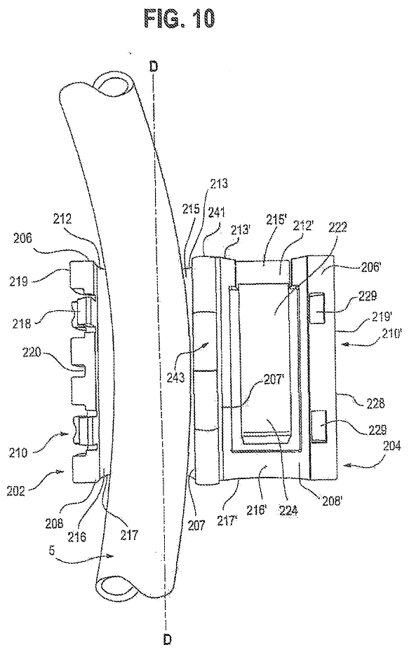

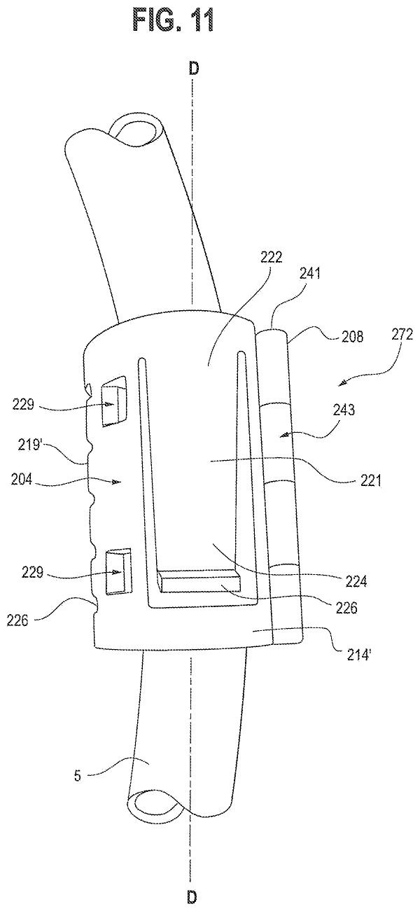

Referring to FIGS. 10 and 11, the collar 272 includes a pair of pivotally interconnected elongate c-shaped cylindrical collar members or collars 202, 204 pivotally interconnected by pin 241 extending through a hinge assembly 243 operatively connected to a respective first edge 207 and 207′ of each of the cylindrical members, as described below. Each cylindrical member is adapted to be positioned within and operatively connected to a respective one of the c-shaped collars or clamshells 275, 276 and extending outwardly away from the patient's face coaxially with the longitudinal axis D-D. Each of the elongate cylindrical members includes first and second ends 206, 208 and 206′, 208′ respectively and a body portion 210, 210′ having an inner surface 212, 212′, an outer surface 214, 214′, upper and lower end portions or edges 215, 215′ and 216, 216′ respectively. Each end portion includes a pair of generally parallel extending upper and lower edge surfaces 213, 213′ and 217, 217′ respectively. Each c-shaped cylindrical member 204, 206 further includes first and second edges or edge surfaces 207, 207′ and 219, 219′ respectively, each of the first and second edges extending intermediate the respective upper and lower edge surfaces 213, 213′ and 217, 217′ in a direction which is parallel to the axis D-D. The outer surface 214 of cylindrical member 202 includes at least one annular flange 218 and structural recess 220 extending radially outwardly from the outer surface 214 and adapted to operatively interact with one of the plurality of structural recesses 292 formed intermediate the substantially uniformly spaced-apart annular flanges 294 positioned axially along the inner surface of clamshell 275. While in the embodiment shown only the outer surface 214 of cylindrical member 202 includes at least one annular flange and structural recess, cylindrical member 204 may also be so structured without departing from the scope of the present invention.

Cylindrical member 204 includes a flexible beam member or flex beam 221 operatively connected at a first end 222 thereof to the second end 206′ of the c-shaped cylindrical member 204. The flexible beam member includes a second end 224 having a radially outwardly extending tab or handle 226 adapted to facilitate manipulation of the member 204 during installation on an airway device and a generally curvilinear inner surface 228 having substantially the same curvature as the curvature of the inner surfaces 212, 212′ of the c-shaped cylindrical members 202, 204 of the interlock collar. The flex beam 221 possesses inherent flexible, spring-like properties so that when the interlock collar is in an open configuration, the second end 224 thereof is urged radially inwardly a preselected distance from the inner surface of the cylindrical member 204 in response to the spring like forces exerted by the beam member thereon. Thus, in operation, when the c-shaped members of the interlock collar are urged in operative engagement with an external surface 218 of an airway device via closure of the clamping member or tower structure 270, the flexible beam member is urged into clamping engagement with the airway device's external surface and is adapted to allow the stabilization system to secure airway devices of different sizes in a patient's airway. The interlock collar further includes a latch mechanism 229 adapted to secure the interlock collar in a closed configuration at a selected position on an airway device. In addition, the inner surfaces of each c-shaped members 202, 204 may be coated with an adhesive material, by way of example and not of limitation, a pressure sensitive adhesive (PSA) or an adhesive tape, or some other suitable material adapted to adhesively engage the outer surface of an airway device. As will be discussed in greater detail below, the adhesive is covered by a protective liner that is peeled away prior to installation of the system on an airway device. In yet another embodiment, the inner surfaces may be textured, for example, like the surface texturing found on a porcupine quill, or it may have micro surface texturing such as a micro suction surface structure or a Sharklet® micropattern to selectively prevent axial motion along the B-B axis of an airway device in one or both axial directions.

In operation the pivotally interconnected clamshells 275 and 276 are closed about the collar 272 into mating contact with one another and are retained in locking engagement by a releasable latch mechanism 238. The radially inwardly extending flanges and recesses 292, 294 on the inner surface of clamshell 275 operatively engage mating recesses and flanges 220, 218 formed on and extending radially outwardly from the outer surface 214 of cylindrical member 202, thereby locking the system to maintain the airway device at the correct insertion depth in a patient's trachea. The latch mechanism releases the collar for either positional adjustment along the axis D-D or removal from the tower structure 270.

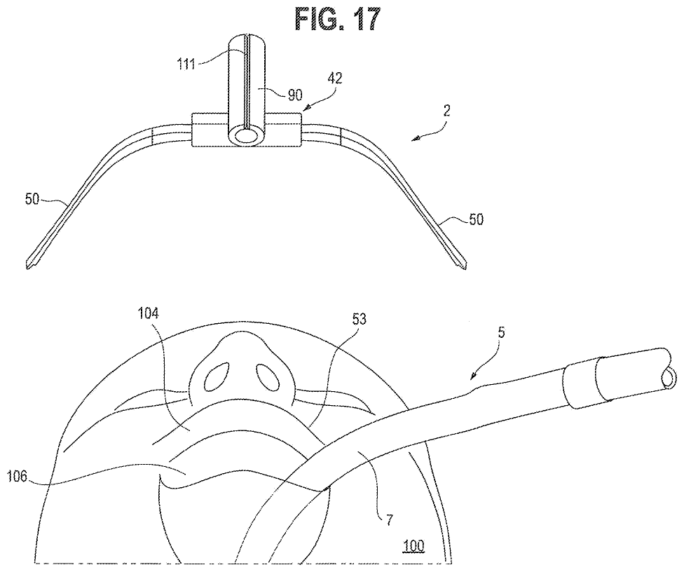

Referring now to FIGS. 16-35, methods are disclosed for intubating and securing an airway device in a patient 100 while the patient is undergoing treatment in a secure, low-risk environment such as an operating room and for transitioning the patient to a more robust airway securement and stabilization system required for higher risk treatments, patient transportation to a different unit in a hospital or to a different facility, or patient movement or manipulation such as proning. The first step, step a, in the process is the insertion of an airway device, for example, an endotracheal tube 5 as shown in FIG. 16, in the patient's mouth 104 and carefully passing it through the patient's oral cavity 106, throat 108, past a patient's vocal cords or larynx 110 into a patient's trachea 112 and to a preselected distance (approximately four centimeters target range 2-6 cm) above a patient's carina (the point where the trachea bifurcates into the bronchial tubes) and inflating a balloon 101. Step b-after the airway device is positioned at the proper depth in the patient's airway, it is moved laterally to the side of the patient's face as shown in FIG. 17, and an airway securement apparatus for use in secure, low risk environments, for example, the apparatus 1 of FIGS. 1-5 or the apparatus 2 of FIGS. 6 and 7, is made ready for placement on the patient, and the patient's skin is cleaned and prepared. For purposes of illustration and not of limitation, airway securement apparatus 2 is shown in FIGS. 17-31.

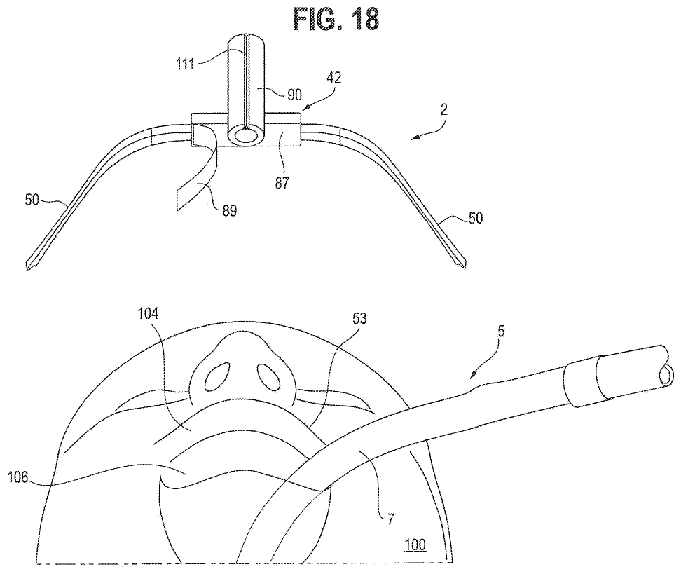

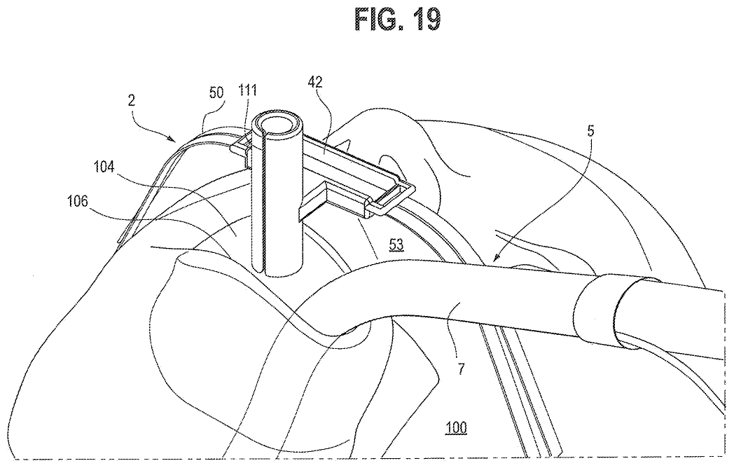

FIG. 18 illustrates the next step, step c, in the installation sequence, namely the removal of a protective backing layer 89 from an adhesive material 87 secured to the bottom of the base 42 of the apparatus. Step d-after the backing layer has been removed, the apparatus 2 is placed on the patient's upper lip 53 and positionally adjusted as needed in the patient's mouth 104 and oral cavity 106, as shown in FIG. 19.

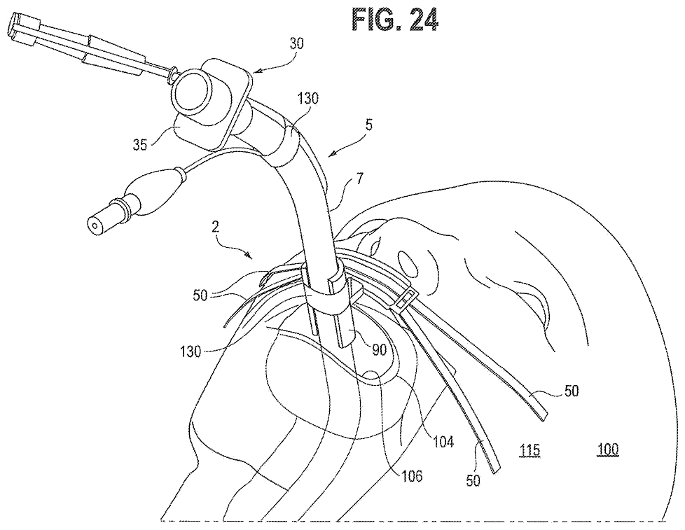

FIG. 20 depicts the next step, step e, which is opening the aperture or slot 111 sufficiently wide to receive the flexible tubular body 7 of the airway device, and thereafter, step f, inserting the tubular body into the tower 90 via the slot 111 (FIG. 21). Step g-securing the airway device is performed by applying a securing or locking device to the tower with the tubular body 7 of the airway device positioned therein. In the embodiment of FIG. 22, the securing step g includes wrapping the tower and the airway device positioned therein with one or more layers of tape 130 and removing the protective backing protective backing layer 54 (FIG. 23) from each of the plurality of securement straps 50 in preparation for placing the straps on a patient's face 115. FIG. 24 illustrates the placement step, step h, of positioning the securement straps 50 in an x-pattern or configuration on the patient's face, which reduces the pressure on the patient's face. The patient then is prepared for receiving the appropriate treatments and/or undergoing the procedures in the operating room prescribed for the patient.

At this point, depending upon the treatment the patient is receiving, the airway device and the securement apparatus may be removed and the patient may be processed out of the health care facility in accordance with standard procedures associated with the type of treatment the patient has received. However, if the patient is to undergo additional procedures that present a higher risk of unintentional extubation, the following additional steps must be taken to secure and stabilize the airway apparatus.

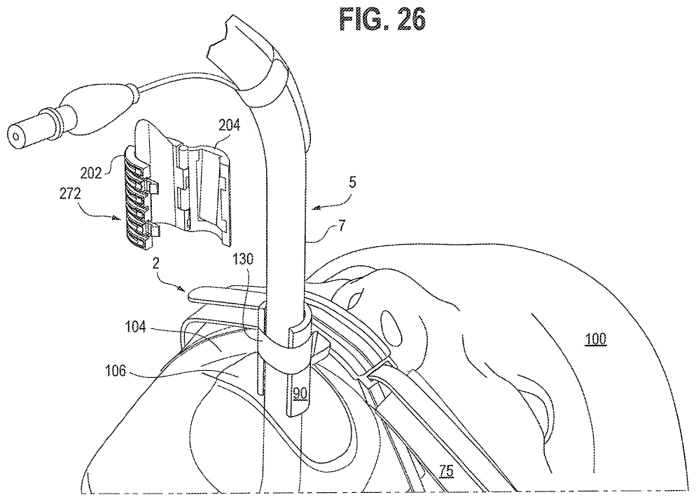

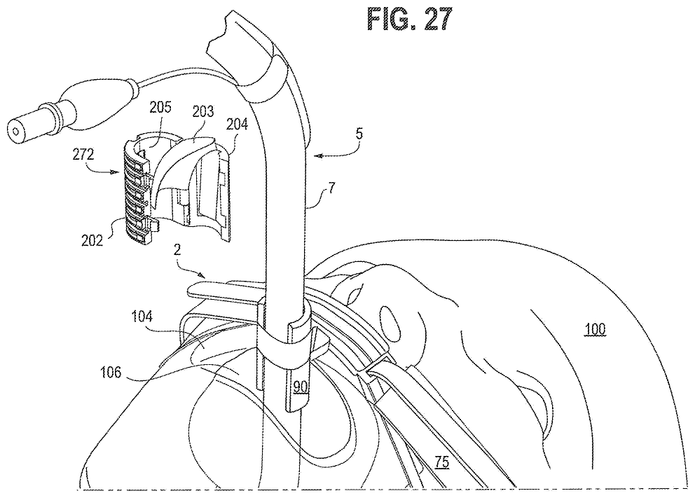

Step i, securing an adjustable safety strap 75 to the cross members 80, 82 located at each end 44, 45 respectively of the base is depicted in FIG. 25. This step is needed only if the patient will undergo high-risk procedures (such as moving the patient from the operating room to the ICU which requires transferring the patient across beds at each location) after receiving treatment in a stable, controlled environment such as an operating room. In that case, additional procedures beginning at step j in FIG. 26 are needed. Step j involves introducing an interlock collar 272 to the process and preparing it for installation on the airway device by first opening the pivotally interconnected collars 202, 204 as shown in FIG. 26. Step k is the step of removing a protective backing film 203 from a layer of pressure sensitive adhesive (PSA) 205 on an inner face of one of the interlock collars. FIG. 27.

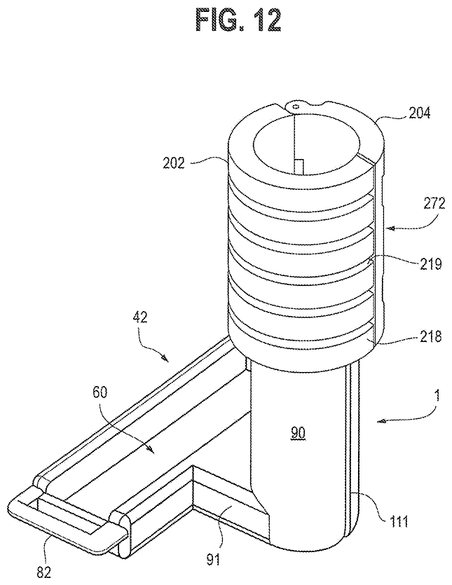

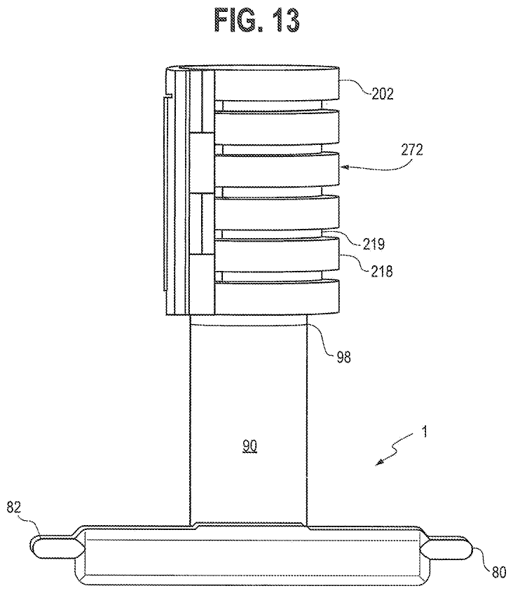

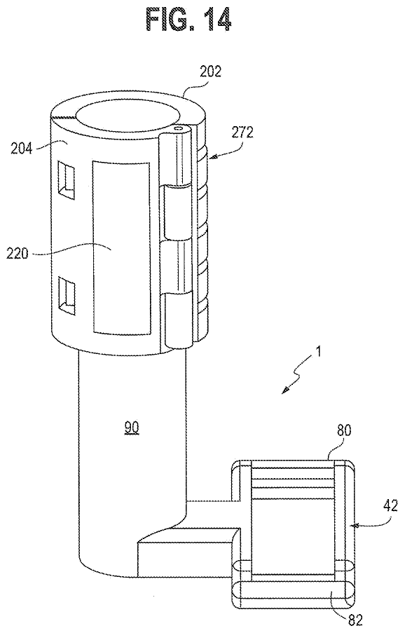

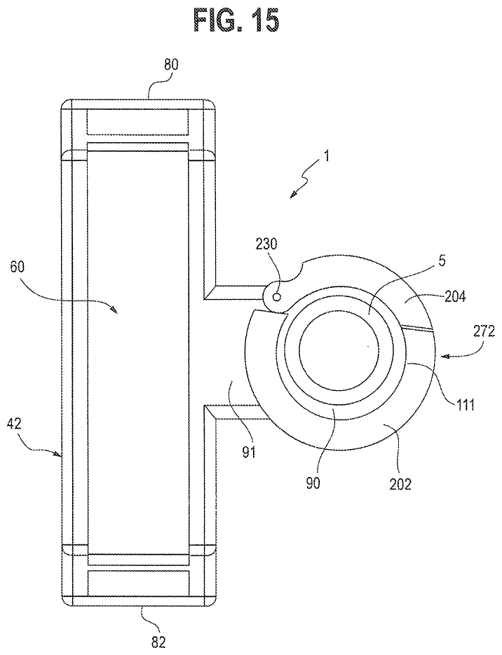

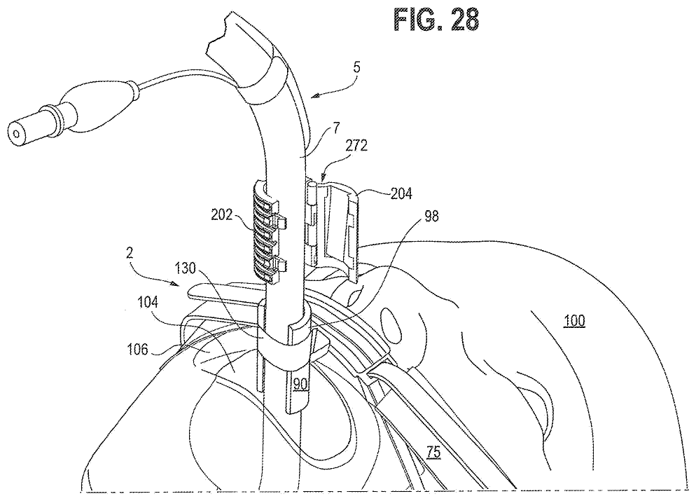

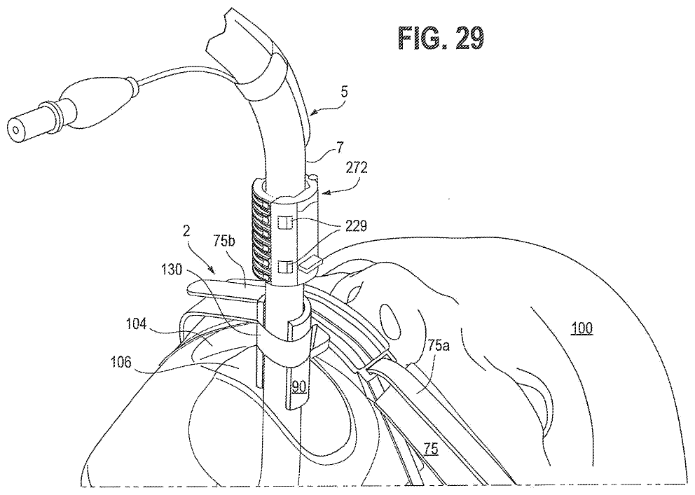

Step l is the step of placing the collar 272 on the body 7 of the airway device at the correct location for transitioning to a more robust securement system. This is done by placing the collar on the top end 98 of the tower 90 as shown in FIG. 28. The collar is secured to the airway device at step m by closing the interlock collar around the airway device until the latches 229 are snapped together in locking engagement, FIG. 29. FIGS. 12-15 and 30 illustrate the interlock collar in the correct position on the top end 98 of the tower 90.

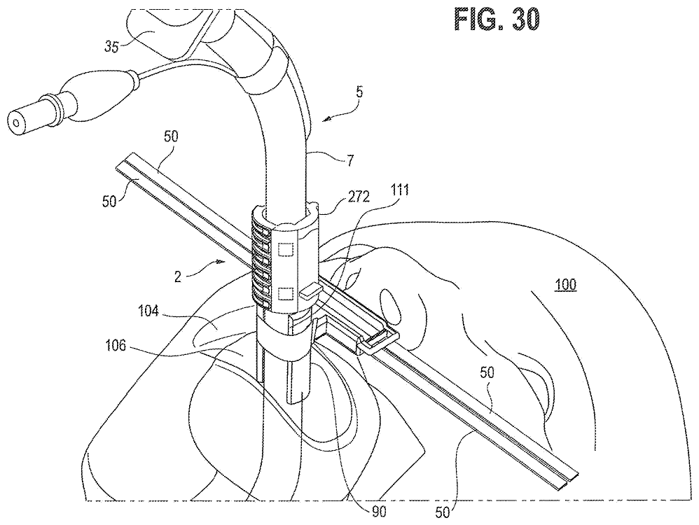

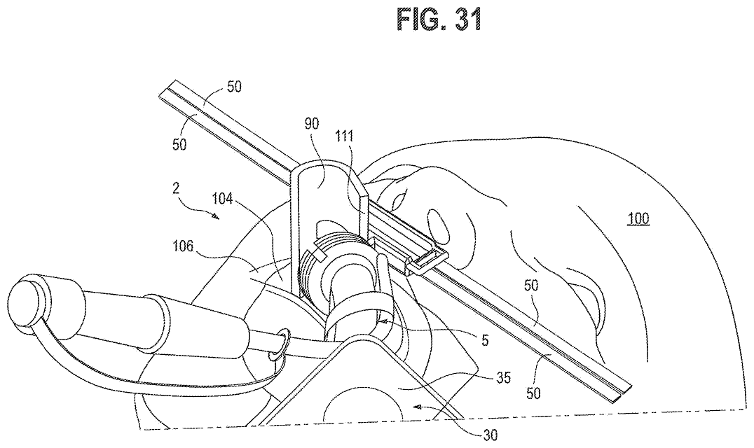

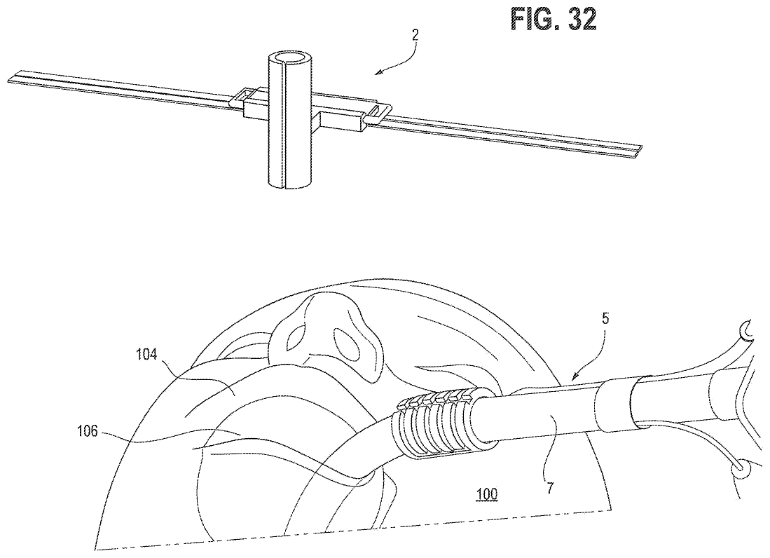

Step n, preparing to remove the airway securement apparatus 2 from the airway device 5 and the patient 100 is shown in FIG. 30. Step n includes a series of sub steps including removing the safety strap 75, detaching each of the securement straps 50 and removing the tape 130 which is wrapped around the tower 90 to secure the airway device 5 therein. Step o shown in FIG. 31 is the step of opening or spreading the aperture 111 sufficiently to permit removal of the airway device therefrom by bending it to the side of the patient's face. Step p, FIG. 32, is the step of physically removing the airway securement apparatus 2 from the patient.

Referring now to FIG. 33, the next step required to establish a more robust securement and stabilization environment for the airway device that remains installed in the patient is the step of preparing the airway stabilization system 200 shown in FIG. 9 for placement on the patient. This step is step q, FIG. 33. Step r as illustrated in FIG. 34 is the step of removing the protective backing 263 from each of the cushioning pads 261, 261′ secured to each end of the stabilization system 200, and step s is the actual placement of the securing system 200 on the patient as shown in FIG. 35 and applying the adhesive cheekpads to the patient's cheeks.

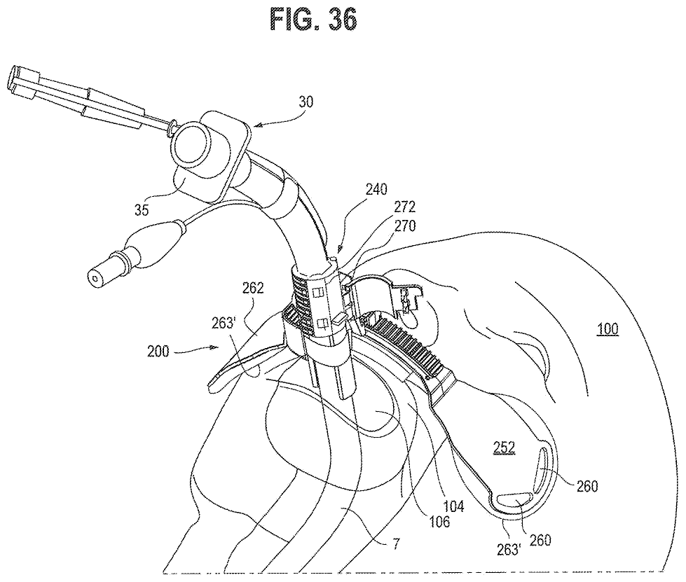

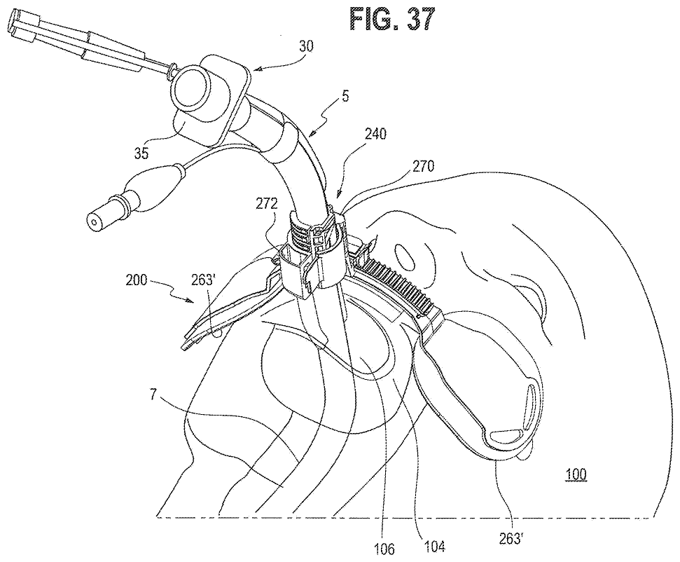

The next step in the installation of system 200 is step t, moving the airway device and the collar 272 from the side of the patient's mouth into the retention structure 240 as shown in FIG. 36. Step u, FIG. 37, is the step of closing the cylindrically-shaped tower structure or clamshell-type clamping member 270 into releasable securing engagement with the interlock collar 272, thereby urging the flexible beam member into clamping engagement with the airway device's external surface. The stabilization system is adapted to allow the system to secure airway devices of different sizes in a patient's airway. The final step in the process, step v, is the step of installing one or more safety straps via apertures 260, 262 formed in each end 261, 261′ respectively of the frame 242 and attaching the straps to the patient.

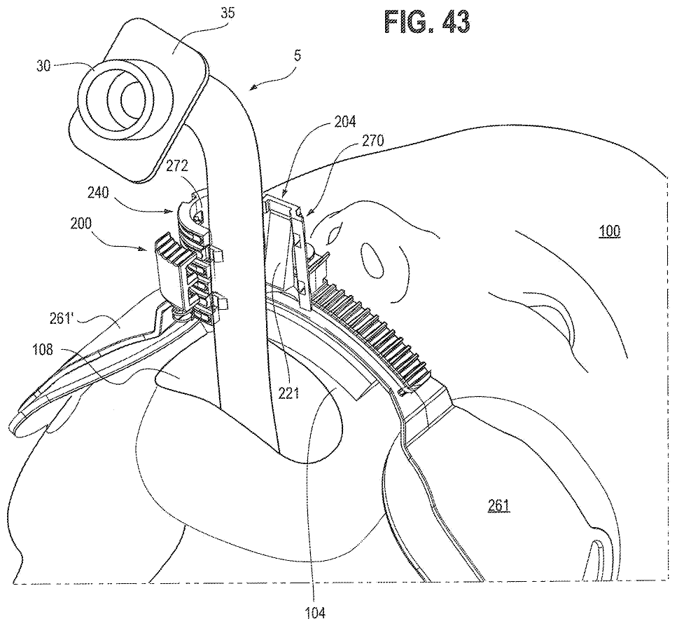

In another embodiment which is illustrated in FIG. 43, the steps of replacing an airway securement apparatus 2 securing an airway device in a patient 100 while the patient is undergoing treatment in a secure, low-risk environment with the airway securement and stabilization system 200 shown in FIG. 9 to establish a more robust securement and stabilization environment for the airway device may be simplified by leaving the interlock collar 272 pre-positioned and properly oriented rotationally within the retention structure 240 of the airway securement and stabilization system 200. At this point in the procedure, a protective backing film 203 has already been removed from a layer of pressure sensitive adhesive or other adhesives suitable for the application 205 on an inner face of the interlock collar at step k above. This step is shown in FIG. 27. Following step p, the step of physically removing the airway securement apparatus 2 from the patient, a next step qq is preparing the airway stabilization system 200 for placement on the patient, as described above. Step rr is the step of removing the protective backing 263 from each of the cushioning pads 261, 261′ secured to each end of the stabilization system 200 in the same manner as shown in FIG. 34 as step r. Step ss is the actual placement of the securing system 200 on the patient in the same manner as shown in step s of the embodiment of FIG. 35 and applying the adhesive cheekpads to the patient's cheeks.

After the securing system 200 is positioned on the patient, at step tt the airway device and the collar 272 is moved from the side of the patient's mouth into the interlock collar 272 which is positioned in the cylindrically shaped tower structure or clamshell-type clamping member 270 of the retention structure 240. Step uu is the step of closing the cylindrically-shaped tower structure or clamshell-type clamping member 270 into releasable securing engagement with the interlock collar 272, thereby urging the flexible beam member into clamping engagement with the airway device's external surface. The final step in the instant embodiment of the process, step vv, is the step of installing one or more safety straps via apertures 260, 262 formed in each end 261, 261′ respectively of the frame 242 and attaching the straps to the patient.

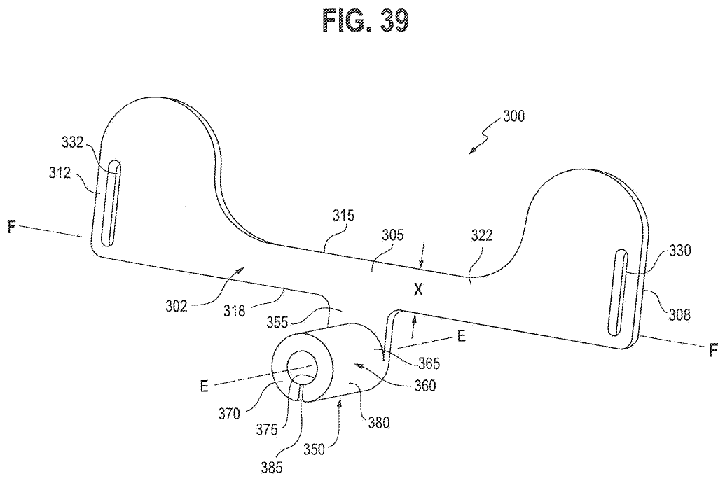

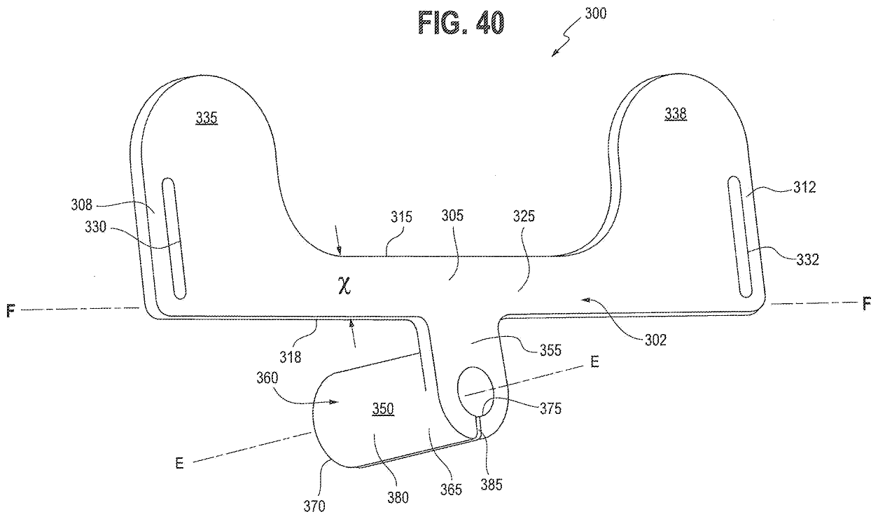

Referring now to FIGS. 39-42, a securing apparatus component 300 of an airway securement apparatus for use in stable environments is illustrated in accordance with another embodiment of the present invention. The securing apparatus 300 includes a base 302 adapted to be secured to a patient and to a collar or tower structure 350 having a longitudinal axis E-E, also referred to herein as simply “the tower”, operatively connected thereto or integrally formed therewith. The base and the tower are formed of the same material, typically a synthetic and preferably silicone; however, as will be described in greater detail below, it is to be understood that other materials having similar properties may be used without departing from the scope of the present invention. The base may be secured to a patient 100 in the same manner as the securing apparatus 42 of the embodiment of FIG. 6 as shown in FIGS. 19-31 by a suitable securement device or attachment mechanism, by way of example and not of limitation, one or more continuous flexible tape strips 50 adapted to be removably affixed or adhered to a patient's face 115 (FIG. 8) or the adjustable strap 75 shown in FIG. 25.

The base 302 includes a rectangular body portion 305 having a width x, the width x of the body portion of the base being sized to fit on an upper lip 53 of a patient. The rectangularly shaped body 305 extends along a longitudinal axis F-F which is perpendicular to axis E-E of the tower, the body having a thickness I, first and second ends 308, 312, first and second sides 315, 318, an upper surface 322 and a lower surface 325, the upper and lower surfaces extending parallel to axis F-F and to one-another intermediate the ends and the sides. Each end 308, 312 includes a connecting point or aperture or slot 330, 332 respectively formed therein extending intermediate the upper and lower surfaces 322, 325 and laterally in a direction perpendicular to the axis F-F and the sides 315, 318. Each connecting point or aperture is adapted to releasably receive an opposing end 75a and 75b respectively of a securement device, for example one or more strap(s) 75 shown in FIGS. 25-29.

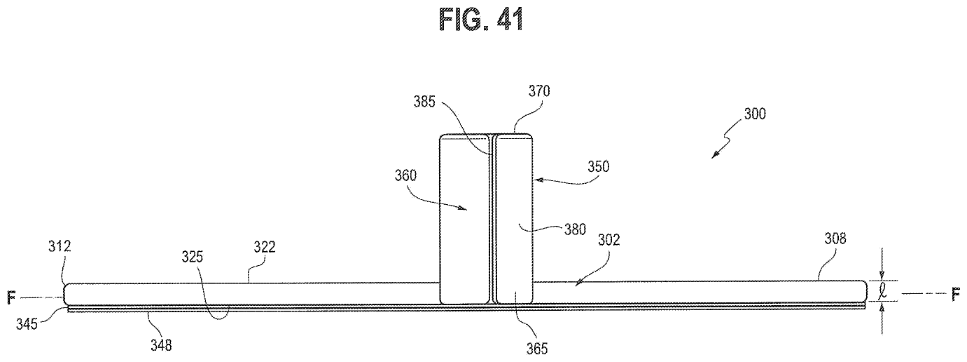

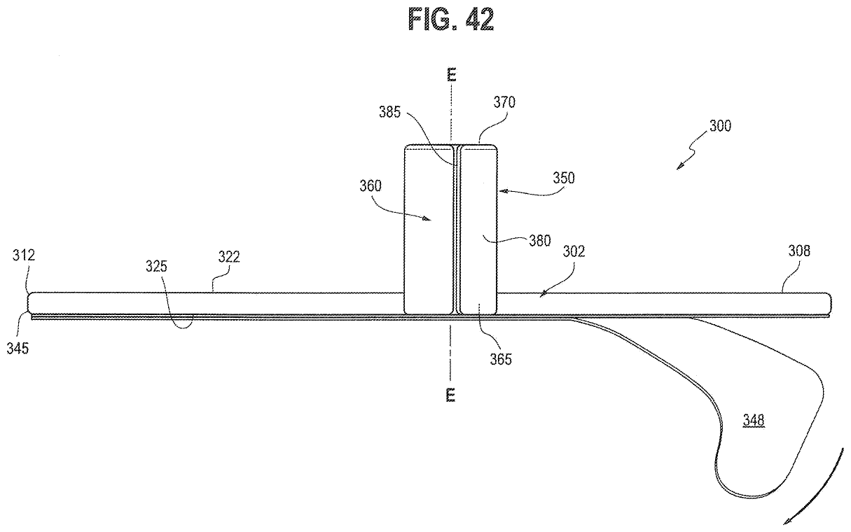

Each end 308, 312 further includes an enlarged cheek pad 335, 338 formed integrally therewith or operatively connected thereto respectively and extending laterally outwardly from the body 305 in a direction perpendicular to both axes E-E and F-F. In the embodiment shown, each cheekpad is semicircular in shape; however, it is to be understood that any shape may be used which is adapted to reduce pressure on a patient's face and to provide additional comfort for the patient. As best viewed in FIGS. 41 and 42, a skin contact hydrocolloid adhesive layer 345 covered by an outer protective layer or film 348 is applied to the lower surface 325 of the body 305 and each of the cheekpads 335 and 338. Prior to installation of the securing apparatus component 300 on a patient, the protective film is peeled away as shown in FIG. 42 exposing the adhesive film. Upon installation of the securement apparatus as described in FIG. 19 et seq., the base and the cheekpads are releasably adhered to a patient's upper lip and cheeks, thereby enhancing the overall apparatus stability.

Similar in structure and operation to the securing apparatus 40 of FIGS. 1-5, the cylindrically shaped tower structure 350 of the securing apparatus 300 in the embodiment depicted in FIGS. 39-42 is operatively connected to or formed integrally with the base 302 via a connecting member 355 extending laterally outwardly from side 318 of the base 302. In one embodiment, the base and the tower are formed of the same material, typically a synthetic and preferably silicone; however, it is to be understood that other materials having properties similar to the properties of synthetic materials or silicone may be used without departing from the scope of the present invention. Moreover, the base and the tower may each be formed of a different material which possesses properties suitable for the intended application, the base and tower being operatively connected via adhesive bonding, threaded fasteners and the like. The tower extends outwardly from the base extends coaxially with the longitudinal axis A-A of an airway device along the longitudinal axis E-E in a direction away from a patient's face. The tower includes a continuous cylindrically-shaped body 360 extending circumferentially about and along the longitudinal axis, the body having a first or bottom end 365 operatively connected to the base and second or top end 370, an inner surface 375 and an outer surface 380, each of the surfaces extending intermediate the top and bottom ends, and an elongate aperture or slot 385 formed in the body intermediate the inner and outer surfaces and extending between the top and bottom ends in a direction parallel to the longitudinal axis E-E. The aperture or slot is adapted to be pried open to fit over the flexible elongate body portions of multiple sizes of airway devices.

In operation, the securing apparatus 300 of the embodiment of FIGS. 39-42 functions in a manner similar to the function of the securing apparatus 40. The apparatus installation and deinstallation steps as set forth above with respect to the patient intubation process using the securing apparatus 40 are identical to the steps of a method of intubation and ventilation a patient using the securing apparatus 300 and are incorporated herein by reference substituting securing apparatus 300 for securing apparatus 40 therein. Thus, coordinating the applications of the novel airway securement and stabilization systems of the present invention, an intubated patient may receive and medical personnel may administer treatments and perform procedures with confidence in a low-risk, stable environments knowing that the risk of unintentional extubation is low. Should the need arise for changing the treatment environment to a scenario where the risk of an unplanned extubation is increased due to either movement of the patient to an ICU, for example, or due to the normal course of treatment, or as a result of unanticipated complications, the airway stabilization apparatus for use in a stable, low-risk environment may be exchanged easily and rapidly for a more robust system using the novel methods herein disclosed.

While only selected embodiments have been chosen to illustrate the present invention, it will be apparent to those skilled in the art from this disclosure that various changes and modifications can be made herein without departing from the scope of the invention as defined in the appended claims. Furthermore, the foregoing descriptions of the embodiments according to the present invention are provided for illustration only, and not for the purpose of limiting the invention as defined by the appended claims and their equivalents.

What is claimed is:

1. An adjustable airway securement apparatus adapted for use in low risk and stabile environments to secure any airway device within a range of sizes that may be used with human patients to maintain an airway in a patient's trachea to a patient's lungs, the airway device including a flexible elongate body extending along a longitudinal axis, the body having a length, an internal diameter and an external diameter, a patient-end portion, a machine end portion, and a continuous sidewall having an internal surface and an external surface extending between the patient end and the machine end portions thereof forming an airway; the patient having a head, a face, a chin, a nose, a chest, a mouth, upper and lower lips, an oral cavity, vocal cords or larynx, a thoracic area, a trachea having a length and forming an airway in the patient, and a carina defining a point at which the trachea separates into a left and a right bronchial tube, the airway securement apparatus comprising:
a base adapted to be secured to a patient;
a collar or tower structure operatively connected to the base and extending outwardly therefrom along a longitudinal axis which extends coaxially with the longitudinal axis of the airway device in a direction away from a patient's face; and
an attachment mechanism operatively connected to the base and adapted to secure the airway securement apparatus to the patient.

2. The adjustable airway securement apparatus of claim 1 wherein the base includes a rectangular body portion extending along a longitudinal axis which is perpendicular to the longitudinal axis of the collar or tower structure, the rectangular body portion having a width adapted to fit on the patient's upper lip.

3. The adjustable airway securement apparatus of claim 2 wherein the rectangular body portion has a thickness, first and second ends, first and second sides, an upper surface, and a lower surface, the upper and lower surfaces extending parallel to the longitudinal axis of the base and to one-another intermediate the ends and the sides, each end including a connector secured thereto and adapted to connect the attachment mechanism to the base.

4. The adjustable airway securement apparatus of claim 3 wherein the attachment mechanism comprises an adjustable safety strap having first and second ends, the adjustable safety strap being operatively connected at the first end thereof to the connector secured to the first end of the body portion of the base and at the second end thereof to the connector secured to the second end of the body portion of the base.

5. The adjustable airway securement apparatus of claim 4 wherein each of the first and second ends of the rectangular body portion includes an enlarged cheek pad operatively connected thereto, each enlarged cheek pad extending laterally outwardly from the body in a direction perpendicular to the longitudinal axis of the collar or tower structure and parallel to the longitudinal axis of the rectangular body portion.

6. The adjustable airway securement apparatus of claim 3 wherein the lower surface of the rectangular body portion of the base includes a skin contact adhesive layer secured thereto, the adhesive layer having a removable overlaying backing or protective layer or film positioned thereon.

7. The adjustable airway securement apparatus of claim 6 wherein the adjustable airway securement apparatus includes a skin contact adhesive operatively connected to the lower surface of the body and each of the cheekpads.

8. The adjustable airway securement apparatus of claim 7 wherein the adjustable airway securement apparatus includes an outer protective layer or film covering the skin contact adhesive operatively connected to the lower surface of the body and each of the cheekpads.

9. The adjustable airway securement apparatus of claim 1 wherein the collar or tower structure and the base are integrally formed together of the same material.

10. The adjustable airway securement apparatus of claim 9 wherein the collar or tower structure and the base are integrally formed of silicone material.

11. The adjustable airway securement apparatus of claim 1 wherein the collar or tower structure and the base are integrally formed together of dissimilar materials.

12. The adjustable airway securement apparatus of claim 1 wherein the collar or tower structure and the base are each formed of a different material, the collar and the tower structure being operatively connected to one another.

13. The adjustable airway securement apparatus of claim 1 wherein the tower includes a continuous cylindrically-shaped body extending circumferentially about and along the longitudinal axis, the tower having a first or bottom end operatively connected to the base and second or top end, an inner surface, and an outer surface, each of the surfaces extending intermediate the top and bottom ends, the cylindrically-shaped body further including an elongate aperture or slot formed therein intermediate the inner and outer surfaces and extending between the top and bottom ends in a direction parallel to the longitudinal axis.

14. The adjustable airway securement apparatus of claim 13 further including a connecting member extending laterally outwardly from the second side of the base and operatively connected to the first or bottom end of the tower.

15. The adjustable airway securement apparatus of claim 14 wherein the base further includes a channel formed in the upper surface thereof intermediate the base's first and second ends and first and second sides.

16. The adjustable airway securement apparatus of claim 15 wherein the attachment mechanism comprises one or more continuous flexible tape strips adapted to be removably affixed or adhered to the patient's face, each of the one or more tape strips including an upper surface, a lower surface, first and second ends, and a midsection.

17. The adjustable airway securement apparatus of claim 16 wherein the midsection of each of the one or more tape strips includes an upper surface and a lower surface, the lower surface having an adhesive material suitable for use in medical applications operatively connected thereto, the adhesive material being adapted to operatively connect the one or more tape strips to the channel of the base.

18. The adjustable airway securement apparatus of claim 17 wherein the lower surface of each of the one or more tape strips includes an adhesive layer and a removable backing strip covering the adhesive layer.

19. The adjustable airway securement apparatus of claim 18 wherein the adhesive layer comprises a non-irritating adhesive suitable for medical applications.

20. The adjustable airway securement apparatus of claim 18 wherein the tower structure includes a bottom end portion that extends a preselected distance from the bottom of the connecting member in a direction toward a patient when the apparatus is installed, thereby forming a bite block.

21. A method of maintaining precise, safe and effective intubation and ventilation of a patient in a stable environment and while transitioning from the stable environment to a less stable environment requiring a more robust airway securement and stabilization system, the patient having a head, a face, a chin, a nose, a chest, a mouth, upper and lower lips, an oral cavity, a throat, vocal cords or larynx, a thoracic area, a trachea having a length and forming an airway in the patient, and a carina defining a point at which the trachea separates into a left and a right bronchial tube, the method comprising the steps of:
a. inserting an airway device in a patient's mouth and carefully passing it through the patient's oral cavity, throat, past the patient's vocal cords or larynx into the patient's trachea to a preselected distance (approximately four centimeters target range 2-6 cm) above the patient's carina;
b. moving the airway device laterally to the side of the patient's face;
c. preparing an airway securement apparatus for use in secure, low risk environments for placement on the patient,
d. preparing the patient for placement of the securement apparatus;
e. removing a protective backing layer from an adhesive material secured to a bottom side of a base portion of the securement apparatus;
f. placing the securement apparatus on the patient's upper lip and positionally adjusting it as needed in the patient's mouth and oral cavity;
g. opening an aperture or slot in a collar or tower portion of the securing apparatus sufficiently wide to receive a flexible tubular body portion of the airway device;
h. inserting the tubular body into the tower via the slot;
i. applying a securing or locking device to the tower;
j. removing a protective backing layer from each of one or more securement straps operatively connected to the securement apparatus;
k positioning the securement straps on the patient's face;
l. assessing the patient's post-procedure condition and either, removing the airway device and the securement apparatus; and processing the patient out of the health care facility in accordance with standard procedures associated with the type of treatment the patient has received or if the assessment of the patient's condition indicates that additional procedures are required that present a higher risk of unintentional extubation, then next;
m. securing an adjustable safety strap to the base portion of the airway securement apparatus;
n. preparing a more robust airway securement and stabilization system for placement on the patient;
o. preparing to remove the airway securement apparatus from the airway device and the patient;
p. removing the adjustable safety strap from the base portion of the airway securement apparatus;
q. detaching each of the securement straps from the patient's face;
r. removing the securing or locking device from the tower;
s. opening or spreading the aperture or slot in the collar or tower portion of the securing apparatus sufficiently to permit removal of the airway device therefrom;
t. removing the airway device from the tower portion of the airway securing apparatus by bending it to the side of the patient's face;
u. physically removing the airway securement apparatus from the patient;
v. removing a protective backing from each of a pair of cushioning pads secured to a respective end of the more robust airway securement and stabilization system;
w. placing the securement and stabilization system on the patient and applying the adhesive cheekpads to the patient's cheeks;
x. moving the airway device from the side of the patient's mouth into an interlock collar which is positioned in a cylindrically shaped tower structure or clamshell-type clamping member of a retention structure portion of the airway securement and stabilization system;
y. closing a cylindrically-shaped tower structure or clamshell-type clamping member into releasable securing engagement with the interlock collar, thereby urging a flexible beam member into clamping engagement with the airway device's external surface; and
Z. connecting one or more safety straps to the airway securement and stabilization system and attaching the straps to the patient.

22. A method of maintaining precise, safe and effective intubation and ventilation of a patient in a stable environment and while transitioning from the stable environment to a less stable environment requiring a more robust airway securement and stabilization system, the patient having a head, a face, a chin, a nose, a chest, a mouth, upper and lower lips, an oral cavity, a throat, vocal cords or larynx, a thoracic area, a trachea having a length and forming an airway in the patient, and a carina defining a point at which the trachea separates into a left and a right bronchial tube, the method comprising the steps of:
a. inserting an airway device in a patient's mouth and carefully passing it through the patient's oral cavity, throat, past the patient's vocal cords or larynx into the patient's trachea to a preselected distance (approximately four centimeters target range 2-6 cm) above the patient's carina;
b. moving the airway device laterally to the side of the patient's face;
c. preparing an airway securement apparatus for use in secure, low risk environments for placement on the patient,
d. preparing the patient for placement of the securement apparatus;
e. removing a protective backing layer from an adhesive material secured to a bottom side of a base portion of the securement apparatus;
f. placing the securement apparatus on the patient's upper lip and positionally adjusting it as needed in the patient's mouth and oral cavity;
g. opening an aperture or slot in a collar or tower portion of the securing apparatus sufficiently wide to receive a flexible tubular body portion of the airway device;
h. inserting the tubular body into the tower via the slot;
i. applying a securing or locking device to the tower;
j. removing a protective backing layer from each of one or more securement straps operatively connected to the securement apparatus;
k. positioning the securement straps on the patient's face;
l. assessing the patient's post-procedure condition and either removing the airway device and the securement apparatus; and processing the patient out of the health care facility in accordance with standard procedures associated with the type of treatment the patient has received, or, if the assessment of the patient's condition indicates that additional procedures are required that present a higher risk of unintentional extubation, then next;
m. securing an adjustable safety strap to the base portion of the airway securement apparatus;
n. introducing an interlock collar and preparing it for installation on the airway device by first opening a pair of pivotally interconnected collar portions thereof;
o. removing a protective backing film from a layer of pressure sensitive adhesive or other suitable adhesive on an inner face of one of the interlock collars;
p. placing the interlock collar on the top end of the tower and closing the interlock collar around the body of the airway device until a pair of latches operatively connected thereto are snapped together in locking engagement;
q. preparing to remove the airway securement apparatus from the airway device and the patient;
r. removing the adjustable safety strap from the base portion of the airway securement apparatus;
s. detaching each of the securement straps from the patient's face;
t. removing the securing or locking device from the tower;
u. opening or spreading the aperture or slot collar or tower portion of the securing apparatus sufficiently to permit removal of the airway device therefrom;
v. removing the airway device from the tower portion of the airway securing apparatus by bending it to the side of the patient's face;
w. physically removing the airway securement apparatus from the patient;
x. preparing a more robust airway securement and stabilization system for placement on the patient;
y. removing a protective backing from each of a pair of cushioning pads secured to a respective end of the airway securement and stabilization system;
z. placing the more robust securement and stabilization system on the patient and applying the adhesive cheekpads to the patient's cheeks;
aa. moving the airway device and the interlock collar from the side of the patient's mouth into a cylindrically-shaped tower or retention structure portion of the airway securement and stabilization system;
bb. closing a cylindrically-shaped tower structure or clamshell-type clamping member into releasable securing engagement with the interlock collar, thereby urging a flexible beam member into clamping engagement with the airway device's external surface; and
cc. connecting one or more safety straps to the airway securement and stabilization system and attaching the straps to the patient.

23. A method of maintaining precise, safe and effective intubation and ventilation of a patient in a stable environment and while transitioning from the stable environment to a less stable environment requiring a more robust airway securement and stabilization system, the patient having a head, a face, a chin, a nose, a chest, a mouth, upper and lower lips, an oral cavity, a throat, vocal cords or larynx, a thoracic area, a trachea having a length and forming an airway in the patient, and a carina defining a point at which the trachea separates into a left and a right bronchial tube, the method comprising the steps of:
a. inserting an airway device in a patient's mouth and carefully passing it through the patient's oral cavity, throat, past the patient's vocal cords or larynx into the patient's trachea to a preselected distance (approximately four centimeters target range 2-6 cm) above the patient's carina;
b. moving the airway device laterally to the side of the patient's face;
c. preparing an airway securement apparatus for use in secure, low risk environments for placement on the patient, the securement apparatus including a collar or tower structure operatively connected to a base, the base including rectangular body portion having an upper or top surface and a lower or bottom surface, first and second ends, each end including an enlarged cheek pad operatively connected thereto, the lower surface and each of the cheekpads having a skin contact adhesive or other suitable adhesive layer covered by an outer protective layer or film applied thereto,
d. preparing the patient for placement of the securement apparatus;
e. removing the outer protective layer from the adhesive material on the lower surface and each of the cheekpads of the rectangular body portion of the base;
f. placing the securement apparatus on the patient's upper lip and cheeks and positionally adjusting the collar or tower portion thereof as needed in the patient's mouth and oral cavity;
g. opening an aperture or slot in the collar or tower portion of the securing apparatus sufficiently wide to receive a flexible tubular body portion of the airway device;
h. inserting the tubular body portion of the airway device into the tower via the slot;
i. applying a securing or locking device to the tower;
j. assessing the patient's post-procedure condition and either, removing the airway device and the securement apparatus; and processing the patient out of the health care facility in accordance with standard procedures associated with the type of treatment the patient has received or if the assessment of the patient's condition indicates that additional procedures are required that present a higher risk of unintentional extubation, then next;
k. securing an adjustable safety strap to the base portion of the airway securement apparatus;
l. introducing an interlock collar and preparing it for installation on the airway device by first opening a pair of pivotally interconnected collar portions thereof;
m. removing a protective backing film from a layer of pressure sensitive adhesive or other suitable adhesive on an inner face of one of the interlock collars;
n. placing the interlock collar on the top end of the tower and closing the interlock collar around the body of the airway device until a pair of latches operatively connected thereto are snapped together in locking engagement;
o. preparing to remove the airway securement apparatus from the airway device and the patient;
p. removing the adjustable safety strap from the base portion of the airway securement apparatus;
q. removing the securing or locking device from the tower;
r. opening or spreading the aperture or slot collar or tower portion of the securing apparatus sufficiently to permit removal of the airway device therefrom;
s. removing the airway device from the tower portion of the airway securing apparatus by bending it to the side of the patient's face;
t. physically removing the airway securement apparatus from the patient;
u. preparing a more robust airway securement and stabilization system for placement on the patient;
v. removing a protective backing from each of a pair of cushioning pads secured to a respective end of the airway securement and stabilization system;
w. placing the more robust securement and stabilization system on the patient and applying the adhesive cheekpads to the patient's cheeks;
x. moving the airway device and the interlock collar from the side of the patient's mouth into a cylindrically-shaped tower or retention structure portion of the airway securement and stabilization system;
y. closing a cylindrically-shaped tower structure or clamshell-type clamping member into releasable securing engagement with the interlock collar, thereby urging a flexible beam member into clamping engagement with the airway device's external surface; and
z. connecting one or more safety straps to the airway securement and stabilization system and attaching the straps to the patient.

24. A method of maintaining precise, safe and effective intubation and ventilation of a patient in a stable environment and while transitioning from the stable environment to a less stable environment requiring a more robust airway securement and stabilization system, the patient having a head, a face, a chin, a nose, a chest, a mouth, upper and lower lips, an oral cavity, a throat, vocal cords or larynx, a thoracic area, a trachea having a length and forming an airway in the patient, and a carina defining a point at which the trachea separates into a left and a right bronchial tube, the method comprising the steps of:
a. inserting an airway device in a patient's mouth and carefully passing it through the patient's oral cavity, throat, past the patient's vocal cords or larynx into the patient's trachea to a preselected distance (approximately four centimeters target range 2-6 cm) above the patient's carina;
b. moving the airway device laterally to the side of the patient's face;
c. preparing an airway securement apparatus for use in secure, low risk environments for placement on the patient, the securement apparatus including a collar or tower structure operatively connected to a base, the base including rectangular body portion having an upper or top surface and a lower or bottom surface, first and second ends, each end including an enlarged cheek pad operatively connected thereto, the lower surface and each of the cheekpads having a skin contact adhesive or other suitable adhesive layer covered by an outer protective layer or film applied thereto,
d. preparing the patient for placement of the securement apparatus;
e. removing the outer protective layer from the adhesive material on the lower surface and each of the cheekpads of the rectangular body portion of the base;
f. placing the securement apparatus on the patient's upper lip and cheeks and positionally adjusting the collar or tower portion thereof as needed in the patient's mouth and oral cavity;
g. opening an aperture or slot in a collar or tower portion of the securing apparatus sufficiently wide to receive a flexible tubular body portion of the airway device;
h. inserting the tubular body into the tower via the slot;
i. applying a securing or locking device to the tower;
j. assessing the patient's post-procedure condition and either, removing the airway device and the securement apparatus; and processing the patient out of the health care facility in accordance with standard procedures associated with the type of treatment the patient has received or if the assessment of the patient's condition indicates that additional procedures are required that present a higher risk of unintentional extubation, then next;
k. securing an adjustable safety strap to the base portion of the airway securement apparatus;
l. preparing a more robust airway securement and stabilization system for placement on the patient;
m. preparing to remove the airway securement apparatus from the airway device and the patient;
n. removing the adjustable safety strap from the base portion of the airway securement apparatus;
o. removing the securing or locking device from the tower;
p. opening or spreading the aperture or slot in the collar or tower portion of the securing apparatus sufficiently to permit removal of the airway device therefrom;
q. removing the airway device from the tower portion of the airway securing apparatus by bending it to the side of the patient's face;
r. physically removing the airway securement apparatus from the patient;
s. removing a protective backing from each of a pair of cushioning pads secured to a respective end of the more robust airway securement and stabilization system;
t. placing the securement and stabilization system on the patient and applying the adhesive cheekpads to the patient's cheeks;
u. removing a protective backing film from a layer of pressure sensitive adhesive or other suitable adhesive on an inner face of one of the interlock collars;
v. moving the airway device from the side of the patient's mouth into an interlock collar which is positioned in a cylindrically shaped tower structure or clamshell-type clamping member of a retention structure portion of the airway securement and stabilization system;
w. closing a cylindrically-shaped tower structure or clamshell-type clamping member into releasable securing engagement with the interlock collar, thereby urging a flexible beam member into clamping engagement with the airway device's external surface; and
x. connecting one or more safety straps to the airway securement and stabilization system and attaching the straps to the patient.
